SEQUENCE LISTING (4 sequences)

SEQ ID NO: 1 - protein, 1106 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 2 - DNA, 5700 bp, Homo sapiens
agcagccagcagtgactgcccgccctatctgggacccaggatcgctctgtgagcaacttggagccagagaggagatcaacaaggaggaggagagagccggcccctcagccctgctgcccagcagcagcctgtgctcgccctgcccaacgcagacagccagacccagggcggcccctctggcggctctgctcctcccgaaggatgcttggggagtgaggcgaagctgggccgctcctctcccctacagcagcccccttcctccatccctctgttctcctgagccttcaggagcctgcaccagtcctgcctgtccttctactcagctgttacccactctgggaccagcagtctttctgataactgggagagggcagtaaggaggacttcctggagggggtgactgtccagagcctggaactgtgcccacaccagaagccatcagcagcaaggacaccatgcggcttccgggtgcgatgccagctctggccctcaaaggcgagctgctgttgctgtctctcctgttacttctggaaccacagatctctcagggcctggtcgtcacacccccggggccagagcttgtcctcaatgtctccagcaccttcgttctgacctgctcgggttcagctccggtggtgtgggaacggatgtcccaggagcccccacaggaaatggccaaggcccaggatggcaccttctccagcgtgctcacactgaccaacctcactgggctagacacgggagaatacttttgcacccacaatgactcccgtggactggagaccgatgagcggaaacggctctacatctttgtgccagatcccaccgtgggcttcctccctaatgatgccgaggaactattcatctttctcacggaaataactgagatcaccattccatgccgagtaacagacccacagctggtggtgacactgcacgagaagaaaggggacgttgcactgcctgtcccctatgatcaccaacgtggcttttctggtatctttgaggacagaagctacatctgcaaaaccaccattggggacagggaggtggattctgatgcctactatgtctacagactccaggtgtcatccatcaacgtctctgtgaacgcagtgcagactgtggtccgccagggtgagaacatcaccctcatgtgcattgtgatcgggaatgaggtggtcaacttcgagtggacatacccccgcaaagaaagtgggcggctggtggagccggtgactgacttcctcttggatatgccttaccacatccgctccatcctgcacatccccagtgccgagttagaagactcggggacctacacctgcaatgtgacggagagtgtgaatgaccatcaggatgaaaaggccatcaacatcaccgtggttgagagcggctacgtgcggctcctgggagaggtgggcacactacaatttgctgagctgcatcggagccggacactgcaggtagtgttcgaggcctacccaccgcccactgtcctgtggttcaaagacaaccgcaccctgggcgactccagcgctggcgaaatcgccctgtccacgcgcaacgtgtcggagacccggtatgtgtcagagctgacactggttcgcgtgaaggtggcagaggctggccactacaccatgcgggccttccatgaggatgctgaggtccagctctccttccagctacagatcaatgtccctgtccgagtgctggagctaagtgagagccaccctgacagtggggaacagacagtccgctgtcgtggccggggcatgccccagccgaacatcatctggtctgcctgcagagacctcaaaaggtgtccacgtgagctgccgcccacgctgctggggaacagttccgaagaggagagccagctggagactaacgtgacgtactgggaggaggagcaggagtttgaggtggtgagcacactgcgtctgcagcacgtggatcggccactgtcggtgcgctgcacgctgcgcaacgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttacgagatccgatggaaggtgattgagtctgtgagctctgacggccatgagtacatctacgtggaccccatgcagctgccctatgactccacgtgggagctgccgcgggaccagcttgtgctgggacgcaccctcggctctggggcctttgggcaggtggtggaggccacggctcatggcctgagccattctcaggccacgatgaaagtggccgtcaagatgcttaaatccacagcccgcagcagtgagaagcaagcccttatgtcggagctgaagatcatgagtcaccttgggccccacctgaacgtggtcaacctgttgggggcctgcaccaaaggaggacccatctatatcatcactgagtactgccgctacggagacctggtggactacctgcaccgcaacaaacacaccttcctgcagcaccactccgacaagcgccgcccgcccagcgcggagctctacagcaatgctctgcccgttgggctccccctgcccagccatgtgtccttgaccggggagagcgacggtggctacatggacatgagcaaggacgagtcggtggactatgtgcccatgctggacatgaaaggagacgtcaaatatgcagacatcgagtcctccaactacatggccccttacgataactacgttccctctgcccctgagaggacctgccgagcaactttgatcaacgagtctccagtgctaagctacatggacctcgtgggcttcagctaccaggtggccaatggcatggagtttctggcctccaagaactgcgtccacagagacctggcggctaggaacgtgctcatctgtgaaggcaagctggtcaagatctgtgactttggcctggctcgagacatcatgcgggactcgaattacatctccaaaggcagcacctttttgcctttaaagtggatggctccggagagcatcttcaacagcctctacaccaccctgagcgacgtgtggtccttcgggatcctgctctgggagatcttcaccttgggtggcaccccttacccagagctgcccatgaacgagcagttctacaatgccatcaaacggggttaccgcatggcccagcctgcccatgcctccgacgagatctatgagatcatgcagaagtgctgggaagagaagtttgagattcggccccccttctcccagctggtgctgcttctcgagagactgttgggcgaaggttacaaaaagaagtaccagcaggtggatgaggagtttctgaggagtgaccacccagccatccttcggtcccaggcccgcttgcctgggttccatggcctccgatctcccctggacaccagctccgtcctctatactgccgtgcagcccaatgagggtgacaacgactatatcatccccctgcctgaccccaaacccgaggttgctgacgagggcccactggagggttcccccagcctagccagctccaccctgaatgaagtcaacacctcctcaaccatctcctgtgacagccccctggagccccaggacgaaccagagccagagccccagcttgagctccaggtggagccggagccagagctggaacagttgccggattcggggtgccctgcgcctcgggcggaagcagaggatagcttcctgtagggggctggcccctaccctgccctgcctgaagctccccccctgccagcacccagcatctcctggcctggcctgaccgggcttcctgtcagccaggctgcccttatcagctgtccccttctggaagctttctgctcctgacgtgttgtgccccaaaccctggggctggcttaggaggcaagaaaactgcaggggccgtgaccagccctctgcctccagggaggccaactgactctgagccagggttcccccagggaactcagttttcccatatgtaaaatgggaaagttaggcttgatgacccagaatctaggattctctccctggctgacaggtggggagaccgaatccctccctgggaagattcttggagttactgaggtggtaaattaacttttttctgttcagccagctacccctcaaggaatcatagctctctcctcgcacttttatccacccaggagctagggaagagaccctagcctccctggctgctggctgagctagggcctagccttgagcagtgttgcctcatccagaagaaagccagtctcctccctatgatgccagtccctgcgttccctggcccgagctggtctggggccattaggcagcctaattaatgctggaggctgagccaagtacaggacacccccagcctgcagcccttgcccagggcacttggagcacacgcagccatagcaagtgcctgtgtccctgtccttcaggcccatcagtcctggggctttttctttatcaccctcagtcttaatccatccaccagagtctagaaggccagacgggccccgcatctgtgatgagaatgtaaatgtgccagtgtggagtggccacgtgtgtgtgccagtatatggccctggctctgcattggacctgctatgaggctttggaggaatccctcaccctctctgggcctcagtttccccttcaaaaaatgaataagtcggacttattaactctgagtgccttgccagcactaacattctagagtattccaggtggttgcacatttgtccagatgaagcaaggccatataccctaaacttccatcctgggggtcagctgggctcctgggagattccagatcacacatcacactctggggactcaggaaccatgccccttccccaggcccccagcaagtctcaagaacacagctgcacaggccttgacttagagtgacagccggtgtcctggaaagcccccagcagctgccccagggacatgggaagaccacgggacctctttcactacccacgatgacctccgggggtatcctgggcaaaagggacaaagagggcaaatgagatcacctcctgcagcccaccactccagcacctgtgccgaggtctgcgtcgaagacagaatggacagtgaggacagttatgtcttgtaaaagacaagaagcttcagatgggtaccccaagaaggatgtgagaggtgggcgctttggaggtttgcccctcacccaccagctgccccatccctgaggcagcgctccatgggggtatggttttgtcactgcccagacctagcagtgacatctcattgtccccagcccagtgggcattggaggtgccaggggagtcagggttgtagccaagacgcccccgcacggggagggttgggaagggggtgcaggaagctcaacccctctgggcaccaaccctgcattgcaggttggcaccttacttccctgggatccccagagttggtccaaggagggagagtgggttctcaatacggtaccaaagatataatcacctaggtttacaaatatttttaggactcacgttaactcacatttatacagcagaaatgctattttgtatgctgttgagtttttctatctgtgtacttttttttaagggaaagattttaatattaaacctggtgcttctcactcaca

SEQ ID NO: 3 - protein, 855 aa, Homo sapiens
MILIPRMLLVLFLLLPILSSAKAQVNPAICRYPLGMSGGQIPDEDITASSQWSESTAAKYGRLDSEEGDGAWCPEIPVEPDDLKEFLQIDLHTLHFITLVGTQGRHAGGHGIEFAPMYKINYSRDGTRWISWRNRHGKQVLDGNSNPYDIFLKDLEPPIVARFVRFIPVTDHSMNVCMRVELYGCVWLDGLVSYNAPAGQQFVLPGGSIIYLNDSVYDGAVGYSMTEGLGQLTDGVSGLDDFTQTHEYHVWPGYDYVGWRNESATNGYIEIMFEFDRIRNFTTMKVHCNNMFAKGVKIFKEVQCYFRSEASEWEPNAISFPLVLDDVNPSARFVTVPLHHRMASAIKCQYHFADTWMMFSEITFQSDAAMYNNSEALPTSPMAPTTYDPMLKVDDSNTRILIGCLVAIIFILLAIIVIILWRQFWQKMLEKASRRMLDDEMTVSLSLPSDSSMFNNNRSSSPSEQGSNSTYDRIFPLRPDYQEPSRLIRKLPEFAPGEEESGCSGVVKPVQPSGPEGVPHYAEADIVNLQGVTGGNTYSVPAVTMDLLSGKDVAVEEFPRKLLTFKEKLGEGQFGEVHLCEVEGMEKFKDKDFALDVSANQPVLVAVKMLRADANKNARNDFLKEIKIMSRLKDPNIIHLLAVCITDDPLCMITEYMENGDLNQFLSRHEPPNSSSSDVRTVSYTNLKFMATQIASGMKYLSSLNFVHRDLATRNCLVGKNYTIKIADFGMSRNLYSGDYYRIQGRAVLPIRWMSWESILLGKFTTASDVWAFGVTLWETFTFCQEQPYSQLSDEQVIENTGEFFRDQGRQTYLPQPAICPDSVYKLMLSCWRRDTKNRPSFQEIHLLLLQQGDE

SEQ ID NO: 4 - DNA, 10166 bp, Homo sapiens
caaaggcatcttgcatcagcctgtggatgtatgcctaccaccgggctccttcaccagcaaagtggaaaaagaagcgtttcacaacaaattcttctttttgggttggggaaacgcagtggattatagctctgttttcttctttccaaaactgtgcacccctggatgaaacctccatcaagggagacctgcaagttgcctggggttcagtgctctagaaagttccaaggtttgtggcttgaattattctaaagaagctgaaataattgaagagaagcagaggccagctgtttttgaggatcctgctccacagagaatgctctgcacccgttgatatgcctcccaggacccagagggagactgtagcctcatttctgtggagacctttggctggactctcctggctctcccagagactccagttccaacaccatcttctgagatgatcctgattcccagaatgctcttggtgctgttcctgctgctgcctatcttgagttctgcaaaagctcaggttaatccagctatatgccgctatcctctgggcatgtcaggaggccagattccagatgaggacatcacagcttccagtcagtggtcagagtccacagctgccaaatatggaaggctggactcagaagaaggggatggagcctggtgccctgagattccagtggaacctgatgacctgaaggagtttctgcagattgacttgcacaccctccattttatcactctggtggggacccaggggcgccatgcaggaggtcatggcatcgagtttgcccccatgtacaagatcaattacagtcgggatggcactcgctggatctcttggcggaaccgtcatgggaaacaggtgctggatggaaatagtaacccctatgacattttcctaaaggacttggagccgcccattgtagccagatttgtccggttcattccagtcaccgaccactccatgaatgtgtgtatgagagtggagctttacggctgtgtctggctagatggcttggtgtcttacaatgctccagctgggcagcagtttgtactccctggaggttccatcatttatctgaatgattctgtctatgatggagctgttggatacagcatgacagaagggctaggccaattgaccgatggtgtgtctggcctggacgatttcacccagacccatgaataccacgtgtggcccggctatgactatgtgggctggcggaacgagagtgccaccaatggctacattgagatcatgtttgaatttgaccgcatcaggaatttcactaccatgaaggtccactgcaacaacatgtttgctaaaggtgtgaagatctttaaggaggtacagtgctacttccgctctgaagccagtgagtgggaacctaatgccatttccttcccccttgtcctggatgacgtcaaccccagtgctcggtttgtcacggtgcctctccaccaccgaatggccagtgccatcaagtgtcaataccattttgcagatacctggatgatgttcagtgagatcaccttccaatcagatgctgcaatgtacaacaactctgaagccctgcccacctctcctatggcacccacaacctatgatccaatgcttaaagttgatgacagcaacactcggatcctgattggctgcttggtggccatcatctttatcctcctggccatcattgtcatcatcctctggaggcagttctggcagaaaatgctggagaaggcttctcggaggatgctggatgatgaaatgacagtcagcctttccctgccaagtgattctagcatgttcaacaataaccgctcctcatcacctagtgaacaagggtccaactcgacttacgatcgcatctttccccttcgccctgactaccaggagccatccaggctgatacgaaaactcccagaatttgctccaggggaggaggagtcaggctgcagcggtgttgtgaagccagtccagcccagtggccctgagggggtgccccactatgcagaggctgacatagtgaacctccaaggagtgacaggaggcaacacatactcagtgcctgccgtcaccatggacctgctctcaggaaaagatgtggctgtggaggagttccccaggaaactcctaactttcaaagagaagctgggagaaggacagtttggggaggttcatctctgtgaagtggagggaatggaaaaattcaaagacaaagattttgccctagatgtcagtgccaaccagcctgtcctggtggctgtgaaaatgctccgagcagatgccaacaagaatgccaggaatgattttcttaaggagataaagatcatgtctcggctcaaggacccaaacatcatccatctattagctgtgtgtatcactgatgaccctctctgtatgatcactgaatacatggagaatggagatctcaatcagtttctttcccgccacgagccccctaattcttcctccagcgatgtacgcactgtcagttacaccaatctgaagtttatggctacccaaattgcctctggcatgaagtacctttcctctcttaattttgttcaccgagatctggccacacgaaactgtttagtgggtaagaactacacaatcaagatagctgactttggaatgagcaggaacctgtacagtggtgactattaccggatccagggccgggcagtgctccctatccgctggatgtcttgggagagtatcttgctgggcaagttcactacagcaagtgatgtgtgggcctttggggttactttgtgggagactttcaccttttgtcaagaacagccctattcccagctgtcagatgaacaggttattgagaatactggagagttcttccgagaccaagggaggcagacttacctccctcaaccagccatttgtcctgactctgtgtataagctgatgctcagctgctggagaagagatacgaagaaccgtccctcattccaagaaatccaccttctgctccttcaacaaggcgacgagtgatgctgtcagtgcctggccatgttcctacggctcaggtcctccctacaagacctaccactcacccatgcctatgccactccatctggacatttaatgaaactgagagacagaggcttgtttgctttgccctcttttcctggtcacccccactccctacccctgactcatatacacttttttttttttttacattaaagaactaaaaaaggaaaaaaaaaagcctagggcagatacaatctagtaaaagaaaatctttgatataccaaagtgttggataacaaaggctagaaaattcagataatttataaaggttaactatacttgtgcttataaatgtgcagattctacaatatttttccatgtcattctaaaagaatcttcagaaagaaaaacttgaagaatactaatgtcttgggaaacatgagattaagtttagggaaaacacttgatagatgtggagaatctgaggactcagaattcagcaactaatcacaggtggttttctaatttggcctctggacatgtctcactgtttgtatttctctctcctgtcaaagtgaatgatatattcttgaaaacccccaatttcttgaaatgggtgttctgttcattcatgggcaggggtcaggattgaagttcatatatgaaacaactggggatgtagataggaaagaatgtctgctgcaagagtgtatgaagggggattgtcatttacaaaaaaaaagtacctcatggatggaaggattcatgaatagataatggacataggaaaggaggtcaatggaggacacataacagacatgctatccaccatttatttgtgattttgtaagagggttctcttctttgtcttgttcagatattttcatgtgtgtgttttagtgagaatctgagtttttaatcagtaaagtctgatgttttgatatcttgatgttttgatggtagactcttgagttcatcttgtccaaatcttctagcattttcaaggatgaagcctctgtgggatctttgggcttggggctgaatttctcaggcataaatacctaatggccttgtctaagagggaatagatctgttctgagtgtccccaaaaaagttgtcctaggaactgatgtggagaagttacagggagactgatttccattccttattggggaaaacgaagcactcagaacatacaaggataaatgggctgccaccagaggtgaggagtctcttgtcactggagaggttaagatagaggctcaagaacaatgcaaggaaaatggaggttttcaaacacaaagtggatggtggtatgagacaatgtttaatgtccttctaactccgagagtccttgattctggagaggcacaggatgaacatctgttgctactgctgagtatacacactcttgcctctgagatggcgtgaccagcagaaaagaaagctcagggctgtggcttcagaattcatggaaacagagccactgtcaagaaggaatctgctcagagcagattctgagttttcacactccacaaatcctcagatttagtgggaagctggaaaaaggaactcttaattccaattgaggagaaacaagaaaacattacaaatcagtttcccaagctgagaaggattagccttgaccccttgggtctgcctctgttgccctcaccagtttaccttctacaacagtttctcaggaacatgtgtatttccccattcagctttcagcattgctccacgccacagcataagcatagatcccaagtccacaggctccattttgcaggtcatcttctgatcctagcaaatgtcctttccccatagttgtcctatgcctttgggctttagtctatcccaggactaactgtggagaaatcattggtttgagagtcaagagagcattggtttgggagctttaatcctctttctgcttcacactaagtgtgtcatcttggctaaatcacttggtctttctgcattttgttttcttatttataggatgaggaaattagattaaatggttttgaggtcctttcttgttctgatatgtccagtactcactggaaaattggatctataactgatgggtttagtaatctggtcatttcttgctctgaaaattgtagtcagcaaaagagatcatggaagaaatcactgtaatggtagtaatagtaacacatgccatttgtattgtgccttaggtttaccaggtgtttccaaatacattagcatatttgatatgtgcaggactagataccttgggacctgccacactccactttcaagatatgtattagcttcattagaattaaagggacttgaactcaggacctgcagcctattcttctttatccacatgtctctggtagggctacatccagatcacaccatgacttcttatagagcaagagaaaataatattattatatcttcctttgcctaaaatctctccacttattcttttttatgattctgcaccagttcactgggttattctatgattccatattttttttaaaaaaaatcatatttaaaatgaacttacaatgtctgaattttcctggccttgagtcacagaagtaatatgtttcagatggctgcccaatatgtattatcatgtaatacatatctgtgtccttttctgggatgaggaaggcttcaacttctggcactgagaactttgtattacagacacaggtttagtttctagcttagtcatgcctttagagtttagtagcacaaatccatgcaacccaacagaatacatggtgagggcctagtatgtagaatttgaaagtagttcaaatttgaattagaagaatgaagctaggacttatttggaaaaggagatagaaaaaaaatgatcagaaactgtggggccttattacctttgcagtaagttatcttcttcatgatatatgtgaattattttatgtgcagattgtgttttgggattgtcagatctaaacttatattcttgctggctaatgtgctgatagccaggtctgaaacttgatgtgctatccagacacatatgatcagaaaagatctagagtgcaaagaggtgcctgaggagcaaaatgtggttttaatgttgtggaaagatcacttgcaagtatataagactgtataaagaagaggactgtgtgcaagtggggtgaaaaaaaaggatacgtgaatgtgcatatgactgaatagggaggaaggtcagggctagaaaggaggctacataaaaaggggcaatggagaatgcacaggaaagacacaggggaaggtcaagtcgagcaaggtagaaacaggagtagctagagccattgggaatccattttgaaacaagaaggagttttgaaagggaataggaaagtaagtgtcttgaagtaaaagataaatatggatggagaaagaagaaattctggatgatagagatgataaaaatatttattaagaaatgaagtcaggttcagtgtatgaaatggaaaggaatttttcagaattttaagaaaggggaagttcctcttggaaaagatatagcaaccatcggggaatgaccttttcatttcagaagtggatgaggaaggtggtgtgagcatcaggtatattctggaccatttcaagtgctggtgagaagaaaggaactctttgcctgaactgggcttggttttccaagtgctgctttggaaatgaagacccagagatgcagagcttatggtagttcataaatcttcatgttctattatctttcatctgccaataaagttcattttcaataatgtccaccattgctgtgcccagaataaccacaggcaaacatcaaaacaatacgcataagttagacaagattaaatcttgtctgatatctgcacaaacagatatgcaccatgttggaaacatgtgttttcctagtcccatccaggcttcccacaagaaagccatgatgtgggtctaaaccatatgttttgagtaaaggagaatagaagaaggggagtgtccgcaaaatggaaagagatgaagatgttccaaggaagtatgctgaaacagaacagtgaatgttttgcccaaaactacaaaaataaaagaaaaaaagaaaattgcaatacatggctactaagtctttgatcataagtcgaatttatagacctggaatttgccatcctagtctttcctttttagtaagacttctgtcctctggcagtgcatatggtaggtctctaatgtttcttcatctccaggaagatgcagatccttatttttgctgggaaatccttctaaatagaaatgtaacatttttataaaaacagattaatgtgtttttcacttagtaaatgttttcaagagctgaattgagaaggaaagagactggagtggttaatggtgatttgatttctggcattctgagttttctgctacaattagctgcattacttggtgccaaagagcagtggggaattgttgagttgctgtatcctttaaaaaaaaacaaaaaacttgttattttgaaagaacttaaggctcacaagatgttacaaaaatagtagagtggccttaccctagatccagttttccccatttataacatttcacttagtccattttcggaaccagaaaattaacattggcataatgctattaactaaactacagaccttttttcaatttcgccagtttttccacacatattcatttagttgctggatacttttaattcttgctgatttgtaaactggccttgcttggatacaacaggaaagatactatctggataaagttctacagttttagagagactattaacacattaatgtgttcctttgtcatgagcaataccctgcctacactgcttctaaattttctgatttgtttggctgtttggcatctgaaacaatccaagacaaacttagaaagattaggcaacacaaaacacagtaagacctgttcatagcttgttgtctagaaaaccaggtagcaggatattctagatgcttcctgctgcttctacgtgagtaggattagcactggggacaaaataaggagtttagagtaaaccagtatttcagtcaagagttagttggcacttagttaatggcactggaaatagcttgtggagagaatagaatacaatggtatagactcctaatgtttgataaaatactattttcagagtggtagagaggttttatttgcctaaatagccgttattaaatggaataacaaccacattagaccaaattaattgcaaacacagcggcaacctggggagaagttgaaactccagttttgtggattacagttttgagttttatgattgacatttttaagtcccctatttaaggggtcaagattataacaatgtgtgtcttactaagtttctaggtcattgtgagcacttgataaatatttgctgaatgttgtttttttgaatgaacataagatagaaacaaaaacttctcatccagttaactagagtgaatgtagggagaattgttttgcttgtaacatggagagtttattttcaagtgaggaaagagaaaaaaattactcagacttgttcctggtgaagtgcattctctgtttgtatactttttgatggagaaattgatctatagaactgcttaattttttgaggcatttagacagcaatgaaaggtagttctccacaggacaccgaatcaaaaggagagaccagactctggcctcatacccagcctatttgaaacaagctatctagtttctcctgcagacaccttgtcaacaacatgcaacagtgtcaggtgccttgcaggaaaataatctgagtcccaagctagcctgtgctcatccacaatcacaatgaacatgtcaaggaagaatttgcagagactcaagggaagcacaatgggataaggtaatcactttcagtgaaaaactgttttcttgaaaacaggcttggacacaattgaaagctggcttcctgcaaacacaccaagagtctgtaatctagcctatccattatatgtcctttattattcatgatatcctattcttctaccttgttgcctggtaactttttctgaggactgagtttctgcagcgatgtggtgcactcttcctgtgatgaggaaacatctgggcccccttctgcaggctttggaagatgatgtgtcttgtcaaggggtaaagggcaaatggatttaatttctgcttaaaactatcatagacgttccaaatagaatatgtaaaatttctctgtattagaaaaagaaacgtgataccaattgtatattttcttttctttatttattctctgtaagtctgtcagatgataaattgtaaataacaatgattaaagagtcatgctactgatggatctccctttctgtataaacagtgccagttctgggctttgtaacctttgctctttatagtctttcattcctggggaagtgatggggcatgggcccagagctggggtgtatgtggtatggacacctgtttgtggggctttccagcaaaggattatttaaatagacctctaacatatgagttgactgtttattgggaagaaagcatcttggtctctgacatatccaaacatacgagacactgggattttacgtcctcacattaattagtccagttctggggaatccagtctagaattaactggtgatcccttatcgttatggttacagacttgtcttgtttgatacacagaaatcctttttaaatccaaatatagtctgtctcagactacctgcacatgcacaaaccaaaaaaaactatgaaaaccagaatttagactcagtcattatactagagattagaatgaaacgaccccaaacaacccatttcttacctggtctctgagaataagtttatacttttagttttctgagaatatttctcagaatatgtgagttttctgagcacatttttcagcatggggtatggtagataaatcacagggccaaggtttggcagggatagatgggatcattgtgtaccctattgttcttctgatttccaggagaacagaatgagcccatgcaaaacataaacttatgatgattaaaaaaaacacacctatccattcactcatcaataaaaacatattatgatggttatcaagctgtgtcctatgagtgataaaatatttgtaaaatataaaattaaatggcatctattttgaactcta

(19) United States
(12) Patent Application Publication
(10) Pub. No.: US 2024/0216370 A1
(43) Pub. Date: Jul. 4, 2024

(54) TREATMENT OF CORNEAL VASCULARISATION

(71) Applicant: VESTLANDETS INNOVASJONSSELSKAP AS, Bergen (NO)

(72) Inventors: Cecilie BREDRUP, Bergen (NO); Ove BRULAND, Bergen (NO); Eyvind RØDAHL, Bergen (NO); Gunnar HOUGE, Bergen (NO)

(21) Appl. No.: 17/800,648

(22) PCT Filed: Feb. 18, 2021
(86) PCT No.: PCT/EP2021/053972

§ 371 Date: Aug. 18, 2022

(30) Foreign Application Priority Data
Feb. 19, 2020 (GB) 2002291.9
Feb. 19, 2020 (GB) 2002294.3

(51) Int. Cl. A61K 31/506; A61K 9/00; A61K 9/14; C12Q 1/6883

(52) U.S. Cl. CPC A61K 31/506; A61K 9/0048; A61K 9/14; C12Q 1/6883; C12Q 2600/156

(57) ABSTRACT

The present invention relates to methods for treating or preventing vascularisation in one or both corneas of a subject. The methods comprise topically administering an effective amount of a composition comprising dasatinib to one or both eyes of the subject. The method is particularly useful for treating subjects with corneal vascularisation associated with ocular pterygium-digital keloid dysplasia (OPDKD), Warburg-Cinotti syndrome (WCS) or Penttinen syndrome, and other conditions caused by activating mutations in PDGFRβ and DDR2.

### REFERENCE TO SEQUENCE LISTING

The sequence listing submitted on Sep. 20, 2023, as an .txt file named “145590-03_Seq_Listing_ST25_corr.txt” created on created on Sep. 11, 2023, and 37.883 bytes in size is hereby incorporated by reference pursuant to 37 C.F.R. § 1.52(c)(5).

The present invention relates to methods for treating or preventing vascularisation in one or both corneas of a subject. The methods comprise topically administering an effective amount of a composition comprising dasatinib to one or both eyes of the subject. The method is particularly useful for treating subjects with corneal vascularisation associated with ocular pterygium-digital keloid dysplasia (OPDKD), Warburg-Cinotti syndrome (WCS) or Penttinen syndrome, and other conditions caused by activating mutations in PDGFRβ and DDR2.

Ocular pterygium-digital keloid dysplasia (OPDKD) is characterised by ingrowth of conjunctiva (pterygium-like) over the cornea; it is also associated with keloid formation on distal digits (fingers and toes)^{1;2} The patients usually get visually impaired in childhood despite treatment. The condition is currently believed to follow an autosomal dominant pattern of inheritance, but the genetic aetiology of this condition is not known.

There is therefore a need for providing methods of treating OPDKD.

The current inventors have now found a mutation in the platelet derived growth factor receptor-beta (PDGFRβ) gene in a family with OPDKD. PDGFRβ is a receptor tyrosine kinase (RTK). The identified mutation (encoding N666Y) leads to increased auto-phosphorylation of the PDGFRβ protein, and hence aberrant activation of this protein. In a different family with OPDKD, a different mutation in the PDGFRβ gene was found (encoding S548Y), also leading to aberrant activation of the protein.

This discovery opens the door to treatment of OPDKD with RTK inhibitors targeting PDGFRβ.

Penttinen syndrome (OMIM #601812) is characterized by premature aging with involvement of multiple organs. Most patients with this syndrome develop corneal vascularization in the first decades of life. Penttinen syndrome is associated with activating mutations within the receptor tyrosine kinase (RTK) domain of PDGFRβ (e.g. V665A, N666S) [1, 2].

Warburg-Cinotti syndrome (WCS, MIM #618175) is characterized by keloid formation, chronic skin ulcers, wasting of subcutaneous tissue, progressive corneal neovascularization, flexion contractures of the fingers and acro-osteolysis.

WCS is an autosomal dominant disease. It is caused by a heterozygous mutation in the DDR2 gene (191311) on chromosome 1q23 (Xu et al., “Recurrent, activating variants in the receptor tyrosine kinase DDR2 cause Warburg-Cinotti syndrome”, Am. J. Hum. Genet. 103: 976-983, 2018). DDR2 is a receptor tyrosine kinase (RTK).

Two different mutations in the DDR2 gene are known to be associated with this syndrome. The two mutations (encoding Y740C and L610P) are both located in the kinase domain. These mutations disturb the auto-inhibition of the kinase so that it is aberrantly activated.

Given the involvement of RTKs in WCS, the use of RTK inhibitors to treat WCS has previously been proposed; and fibroblasts from WCS patients which were treated in vitro with 0.1 μM dasatinib (a RTK inhibitor) for 6 hours showed normalization of the levels of phosphorylated DDR2 (Xu et al., ibid).

There is a need for providing enhanced methods of treating WCS.

Numerous RTK inhibitors are known. For example, dasatinib (sold under the brand name Sprycel®) and imatinib (sold under the brand name Gleevec®) have both been used to treat among other conditions, chronic myelogenous leukaemia and acute lymphoblastic leukaemia. In addition, there are a few case reports of patients with activating mutations in PDGFRβ benefitting from RTK inhibitors.

The inventors therefore expected that orally-administered RTK inhibitors would be usable for the treatment of the above disorders because such inhibitors are known to have systemic effects. Furthermore, in both WCS and OPDKD, there is a high degree of vascularisation in the cornea and hence it was expected that RTK inhibitors would readily be delivered to the cornea.

However, the current inventors have found that this was not the case for OPDKD. There are no reports of patients with WCS treated with systemic RTK inhibitors. The reason for this failure was not immediately clear to the inventors.

The current inventors subsequently found that both the N666Y amino acid substitution in the PDGFRβ protein in OPDKD patients and the Y740C amino acid substitution in the DDR2 protein in WCS patients are temperature-sensitive, leading to increased activation of the proteins at lower temperatures.

With regard to the N666Y substitution in the PDGFRβ protein, increased levels of auto-phosphorylation were seen at 32° C. compared to 37° C. With regard to the Y740C substitution in the DDR2 protein increased levels of auto-phosphorylation were seen at 32° C. compared to 37° C.

It is known that the corneas, toes and fingers are the parts of the body with the lowest temperatures. In typical room temperature (e.g. 20-22° C), corneal temperatures lie around 32-34° C. and fall to around 30° C. as the air temperature reaches 0° C.

The inventors have realised therefore that the reduced and variable temperatures to which the cornea is exposed cause increased levels of activation of the temperature-sensitive mutations in the PDGFRβ and DDR2 genes, and that this exacerbates the symptoms of OPDKD and WCS. This is the likely cause of OPDKD manifesting in the cornea and fingers where the temperature is reduced. Furthermore, although the numbers are low, patients with WCS living in arctic temperatures (Scandinavia and Alaska) have a more aggressive corneal disease than patients living in warmer climates. It is currently believed that such increased levels of activation cannot effectively be treated with orally-administered RTK inhibitors.

However, the application of drugs (e.g. RTK inhibitors) directly to the corneas of such patients, particularly high-doses of such drugs, could be used to reduce or prevent these symptoms of OPDKD and WCS. In patients with manifested corneal changes, this would also reduce the risk of recurrence after surgery (which is now a major problem in treatment of these patients).

It is one object of the invention, therefore, to provide methods of treating disorders such as OPDKD and WCS with topical RTK inhibitors, preferably dasatinib, particularly high-doses of RTK inhibitors, wherein the RTK inhibitors are administered to one or both eyes of the subject.

It is another object of the invention to provide compositions comprising RTK inhibitors, preferably dasatinib, particularly high-doses of RTK inhibitors, for use in treating disorders such as OPDKD and WCS and other conditions with activating mutations in PDGFRβ and DDR2.

It is yet a further object of the invention to provide compositions which are adapted to be administered to the eye, the compositions comprising RTK inhibitors, preferably dasatinib, particularly high-doses of RTK inhibitors.

In one embodiment, the invention provides a topical composition comprising an RTK inhibitor, preferably dasatinib, for use in treating or preventing vascularisation in one or both corneas of a subject, preferably wherein the composition is administered topically to one or both eyes of the subject. In another embodiment, the invention provides a method of treating or preventing vascularisation in one or both corneas of a subject, the method comprising topically administering an effective amount of a topical composition comprising an RTK-inhibitor, preferably dasatinib, to one or both eyes of the subject. In yet another embodiment, the invention provides a use of an RTK inhibitor, preferably dasatinib, in the manufacture of a topical medicament for treating or preventing vascularisation in one or both corneas of a subject.

In another embodiment, the invention provides a pharmaceutical composition comprising a RTK-inhibitor, preferably dasatinib, wherein the pharmaceutical composition is in the form of a topical composition for administration to the eyes. Preferably, the concentration of the RTK inhibitor in the composition is 0.1 to 100 μM. Preferably, the pharmaceutical composition is packaged in a form which is adapted to dispense the pharmaceutical composition into the eye or onto the cornea. Preferably, the form is adapted to dispense one or more eye drops. The form may be, for example, an eye drop bottle, an eye drop dispenser, or an ophthalmic ointment tube.

In yet a further embodiment, the invention provides a method of diagnosing OPDKD in a subject, the method comprising the steps:

(a) detecting, from a biological sample obtained from the subject, whether the subject's PDGFRβ protein has the mutation N666Y or S548Y;

and optionally

(b) diagnosing the subject with OPDKD when the presence of the N666Y and/or S548Y mutation in the biological sample is detected;

and optionally

(c) administering an effective amount of an RTK inhibitor, preferably dasatinib, to the diagnosed patient.

The invention relates to treating or preventing vascularisation in one or both corneas of the subject. The corneas are in the eyes of the subject. Corneal vascularisation is the in-growth of new blood vessels from the pericorneal plexus/limbus into avascular corneal tissue. The terms corneal vascularisation and corneal neovascularisation are used interchangeably herein.

In some embodiments, the vascularisation or neo-vascularisation is associated with or caused by the aberrant expression of a receptor tyrosine kinase (RTK) in one or both eyes (or corneas) of the subject. Receptor tyrosine kinases (RTKs) are high-affinity, cell-surface receptors for many polypeptide growth factors, cytokines, and hormones. Of the 90 unique tyrosine kinase genes identified in the human genome, 58 encode RTK proteins. Mutations in receptor tyrosine kinases may lead to activation of a series of signalling cascades.

As used herein, the term “aberrant” refers to a non-wild-type level of expression (preferably a higher than wild-type level of expression or overexpression) of the RTK protein or the expression of a mutated form of the RTK protein which has a non-wild-type level of activity (compared to the wild-type protein), preferably higher than wild-type level of activity.

In some embodiments, the RTK is PDGFRβ (Platelet Derived Growth Factor Receptor-beta). In other embodiments, the RTK is DDR2 (Discoidin Domain-containing Receptor 2).

In some embodiments, the vascularisation is due to one or more mutations in the PDGFRβ gene, for example, a mutation which leads to increased activation of the PDGFRβ protein. In other embodiments, the vascularisation is due to one or more mutations in the DDR2 gene, for example, a mutation which leads to increased activation of the DDR2 protein. Each of the mutations in the genes referred to herein may independently be homozygous or heterozygous.

In some embodiments, the mutation is temperature-sensitive. In other embodiments, the mutation is not temperature-sensitive. Preferably, the mutation is a temperature-sensitive mutation. As used herein, the term “temperature-sensitive mutation” refers to a mutation whose phenotype changes at different temperature ranges (e.g. that the kinase has a higher degree of activation). The different temperature ranges are generally known as the “permissive temperatures” and the “non-permissive temperatures”.

At non-permissive temperatures, the protein is unstable, ceases to function properly or functions more aberrantly than at the permissive temperature.

When a temperature-sensitive mutant is expressed in a permissive condition, the mutated gene product behaves normally (meaning that the phenotype is not observed) or less aberrantly, even if there is a mutant allele present. In contrast, the non-permissive temperature or restrictive temperature is the temperature at which the mutant phenotype is observed.

In the context of this invention, an aberrant phenotype is seen at both the permissive and non-permissive temperatures. The phenotype seen at the non-permissive temperature is more extreme (i.e. further from the wild-type phenotype) than the permissive phenotype. In the context of this invention, the permissive temperature is higher than the non-permissive temperature. Preferably, the permissive temperature is 34-39° C., more preferably about 37° C. Preferably, the non-permissive temperature is 28-33° C., more preferably about 32° C.

In some embodiments, the mutation is N666Y in the PDGFRβ protein. In other embodiments, the mutation is Y740C in the DDR2 protein. These mutations are known as activating mutations. These mutations are encoded by the PDGFRβ and DDR2 genes, respectively.

Corneal vascularization is also seen in non-temperature-sensitive mutations in DDR2 and PDGFRβ. In other embodiments, therefore, the mutation is not temperature-sensitive. For example, the non-temperature-sensitive mutation may be L610P in DDR2. For example, the non-temperature-sensitive mutation may be in S548Y, N666S, V665A or N666H in PDGFRβ. These mutations are also known as activating mutations.

The amino acid and nucleotide sequences of the wild-type human PDGFRβ protein are given herein in SEQ ID NOs: 1-2. The amino acid and nucleotide sequences of the wild-type human DDR2 protein are given herein in SEQ ID NOs: 3-4. From the sequences of the subject's PDGFRβ and DDR2 genes, the skilled person will readily be able determine if mutations encoding either of the above-mentioned amino acid substitutions or other substitutions are present. DNA alignment programs such as BLAST may be used in this regard to align the subject's and wild-type genes, and corresponding protein sequences.

The subject is preferably a mammal, e.g. a human, monkey, mouse, rat, horse, cow, pig, sheep or goat. Most preferably, the subject is a human. The human may, for example, be 0-10, 10-20, 20-30, 30-40, 40-50, 50-60, 60-70, 70-80, 80-90, 90-100 or above 100 years old.

The invention is particularly useful for treating subjects with corneal vascularisation associated with ocular pterygium-digital keloid dysplasia (OPDKD) or with Warburg-Cinotti syndrome (WCS) or with Penttinen syndrome.

Preferably, the OPDKD or Penttinen syndrome is due to one or more activating mutations in the PDGFRβ gene, for example, a mutation which leads to increased activation of the PDGFRβ protein.

Preferably, the WCS is due to one or more activating mutations in the DDR2 gene, for example, a mutation which leads to increased activation of the DDR2 protein.

In one embodiment, the subject is one who has Warburg-Cinotti syndrome (WCS, MIM #618175), preferably due to the temperature-sensitive mutation Y740C encoded by the DDR2 gene and/or the non-temperature-sensitive mutation L610P encoded by the DDR2 gene.

In another embodiment, the subject is one who has ocular pterygium-digital keloid dysplasia (OPDKD), preferably due to the temperature-sensitive mutation N666Y encoded by the PDGFRβ gene.

In another embodiment, the subject is one who has Penttinen syndrome, preferably due to a non-temperature sensitive mutation such as S548Y, N666S, N666H or V665A encoded by the PDGFRβ gene.

In some embodiments, the method of the invention additionally comprises the step:

(a) detecting, from a biological sample obtained from the subject, whether the subject's PDGFRβ gene has an activating mutation and/or whether the subject's DDR2 gene has an activating mutation.

The method of the invention comprises topically administering an effective amount of a composition comprising an RTK-inhibitor to one or both eyes of the subject. In some embodiments, a composition comprising a RTK-inhibitor may be injected into the sub-conjunctiva or subtenon, or between the vascular pannus and the cornea. In general, both eyes will be affected by the disease or disorder.

The RTK inhibitor may be administered directly to one or both eyes of the subject, as required. The composition comprising a RTK-inhibitor is administered topically, preferably directly into one or both eyes of the subject. For example, the composition comprising a RTK inhibitor may be administered directly into the eye(s) using an eye drop bottle, eye drop dispenser or eye-bath.

As used herein, the term “topically administered” includes topically applying the composition directly into the eye(s) or onto the cornea(s) of the eye(s).

The method of the invention does not encompass the oral administration of an RTK inhibitor to the subject as the sole method of treatment or prevention. However, the method of the invention may be supplemented by the oral administration of an RTK inhibitor to the subject (which may be the same or a different RTK inhibitor), e.g. to treat symptoms of WGS or OPDKD or Penttinen syndrome in the subject other than corneal vascularisation.

Examples of known RTK inhibitors include the following:

| Inhibitor | RTK target |
|---|---|
| Imatinib (Gleevec) | PDGFR, KIT, Abl, Arg |
| Gefitinib (Iressa) | EGFR |
| Erlotinib (Tarceva) | EGFR |
| Sorafenib (Nexavar) | Raf, VEGFR, PDGFR, Flt3, KIT |
| Sunitinib (Sutent) | KIT, VEGFR, PDGFR, Flt3 |
| Dasatinib (Sprycel) | Abl, Arg, KIT, PDGFR, |
| Nilotinib (Tasigna) | Abl, Arg, KIT, PDGFR |
| Lapatinib (Tykerb) | EGFR, ErbB2 |
| Trastuzumab (Herceptin) | ErbB2 |
| Cetuximab (Erbitux) | EGFR |
| Bevacizumab (Avastin) | VEGF |

Other possible inhibitors include, but are not limited to PB1 (DDR2), AZ628 (Raf-1, Raf-B), Derzazntibinib (ARQ-087), RAF709 (all DDR2); and Linifanib (ABT-869), Axitinib, Baw2881(NVP-BAW2881), Cediranib (AZD2171), Toceranib phosphate, Sitravatinib (MGCD516), AZD3229, ON123300, Regorafenib (BAY 73-4506), AZD2932 and CEP-32496 (RXDX-105).

In some embodiments, the RTK inhibitor is one which inhibits activation of PDGFRβ. In some embodiments, the RTK inhibitor is one which inhibits activation of DDR2. Dasatinib and imatinib both inhibit PDGFRβ and DDR2. Most preferably, the RTK inhibitor is dasatinib.

The dosage of RTK inhibitor that provides the appropriate treatment may depend on many factors, including the size and health of the subject. However, persons of ordinary skill in the art will be able to determine the appropriate dosage to use, based on the teachings herein and particularly the desirability of providing a high dose.

It is preferred that an amount of 0.01-100 μMol RTK inhibitor is delivered to one or each eye in a single administration. The amount may be, for example, 0.01-0.1 μM, 0.1-0.5 μMol, 0.5-1.0 μMol, 1.0-5.0 μMol, 5.0-10 μMol, 10-20 μMol, 20-50 μMol or 50-100 μMol.

In order to achieve this, the composition comprising a RTK inhibitor preferably has a concentration of RTK inhibitor of a least 0.01-100 μM, e.g. 0.001-0.01 μM, 0.01-0.1 μM, 0.1-1.0 μM, 1.0-10 μM or 10-100 μM. This may be administered to each eye in an approximate volume of 0.05 ml (1 drop).

The volume to be administered may be 1-10, preferably 1-5, drops. Each drop may be 0.01-0.1 ml, preferably about 0.05 ml. In some embodiments, a single-use formulation is administered to one or each eye. For example, a single-use formulation which comprises 0.01-100 bM of the RTK inhibitor in a 1-10 ml composition.

The RTK inhibitor (preferably dasatinib) may be administered at suitable intervals, e.g. every 2, 3 or 4 hours, per day.

In addition to the RTK inhibitor, the compositions of the invention may additionally comprise one or more other components. The other components may be active or non-active components.

Active components include one or more of steroids, antihistamines, sympathomimetics, beta receptor blockers, parasympathomimetics, parasympatholytics, prostaglandins, nonsteroidal anti-inflammatory drugs (NSAIDs), antibiotics, antifungal, topical anesthetics, an antiallergic, an antiphlogistic, or an agent suitable for lowering intra-ocular pressure.

In some embodiments, the composition does not comprise vinblastine.

The composition comprising an RTK inhibitor may also comprise one or more ophthalmically-acceptable excipients, diluents or carriers.

The composition may also comprise one or more physiologically-compatible vehicles, which the person skilled in the ophthalmic art can select using conventional criteria. The vehicles may be selected from the known ophthalmic vehicles which include, but are not limited to, saline solution, water polyethers such as polyethylene glycol, polyvinyls such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate and salts such as sodium chloride and potassium chloride.

Other additives include carriers, stabilizers, solubilizers, tonicity-enhancing agents, buffer substances, preservatives, thickeners, complexing agents and other excipients. Examples of such additives and excipients can be found in U.S. Pat. Nos. 5,134,124 and 4,906,613.

Carriers used in accordance with the present invention are typically suitable for topical or general administration, and are for example water, mixtures of water and water-miscible solvents, such as C1-C7-alkanols, vegetable oils or mineral oils comprising from 0.5 to 5% by weight hydroxyethylcellulose, ethyl oleate, carboxymethylcellulose, polyvinyl-pyrrolidone and other non-toxic water-soluble polymers for ophthalmic uses, such as, for example, cellulose derivatives, such as methylcellulose, alkali metal salts of carboxymethylcellulose, hydroxymethylcellulose, hydroxyethylcellulose, methyl hydroxypropylcellulose and hydroxypropylcellulose, acrylates or methacrylates, such as salts of polyacrylic acid or ethyl acrylate, polyacrylamides, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. Preferred carriers are water, cellulose derivatives, such as methylcellulose, alkali metal salts of carboxymethylcellulose, hydroxymethylcellulose, hydroxyethylcellulose, methyl hydroxypropylcellulose and hydroxypropylcellulose, neutral Carbopol, or mixtures thereof.

According to one embodiment, the solubilizers used for an ophthalmic composition of the present invention are, for example, tyloxapol, fatty acid glycerol poly-lower alkylene glycol esters, fatty acid poly-lower alkylene glycol esters, polyethylene glycols, glycerol ethers or mixtures of those compounds. The amount added is typically sufficient to solubilize the active ingredient. For example, the concentration of the solubilizer is from 0.1 to 5000 times the concentration of the active ingredient. Lower alkylene means linear or branched alkylene with up to and including 7 C-atoms. Examples are methylene, ethylene, 1,3-propylene, 1,2-propylene, 1,5-pentylene, 2,5-hexylene or 1,7-heptylene. Lower alkylene is preferably linear or branched alkylene with up to and including 4 C-atoms.

Examples of buffer substances are acetate, ascorbate, borate, hydrogen carbonate/carbonate, citrate, gluconate, lactate, phosphate, propionate and TRIS (tromethamine) buffers. Tromethamine and borate buffer are preferred buffers. The amount of buffer substance added is, for example, that necessary to ensure and maintain a physiologically tolerable pH range. The pH range is typically in the range of from 5 to 9, preferably from 6 to 8.2 and more preferably from 6.8 to 8.1.

Tonicity-enhancing agents are, for example, ionic compounds, such as alkali metal or alkaline earth metal halides, such as, for example, CaCl₂, KBr, KCl, LiCl, NaBr, NaCl, or boric acid. Non-ionic tonicity enhancing agents are, for example, urea, glycerol, sorbitol, mannitol, propylene glycol, or dextrose. For example, sufficient tonicity enhancing agent is added to impart to the ready-for-use ophthalmic composition an osmolality of approximately from 50 to 1000 mOsmol, preferred from 100 to 400 mOsmol, more preferred from 200 to 400 mOsmol and even more preferred from 280 to 350 mOsmol.

Examples of preservatives are quaternary ammonium salts, such as cetrimide, benzalkonium chloride or benzoxonium chloride, alkyl-mercury salts of thiosalicylic acid, such as, for example, thimerosal, phenylmercuric nitrate, phenylmercuric acetate or phenylmercuric borate, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, or sorbic acid. Preferred preservatives are cetrimide, benzalkonium chloride, benzoxonium chloride and parabens. Where appropriate, a sufficient amount of preservative is added to the ophthalmic composition to ensure protection against secondary contaminations during use caused by bacteria and fungi.

Such preservatives are typically employed at a level of from 0.001% to 1.0% (w/v) to ensure protection against secondary microbial contaminations during use caused by bacteria, mould, and fungi.

The ophthalmic compositions may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10 000. Other excipients that may be used if desired are listed below but they are not intended to limit in any way the scope of the possible excipients. They are especially complexing agents, such as disodium-EDTA or EDTA, antioxidants, such as ascorbic acid, acetylcysteine, cysteine, sodium hydrogen sulfite, butyl-hydroxyanisole, butyl-hydroxy-toluene or a-tocopherol acetate; stabilizers, such as a cyclodextrin, thiourea, thiosorbitol, sodium dioctyl sulfosuccinate or monothioglycerol; or other excipients, such as, for example, lauric acid sorbitol ester, triethanol amine oleate or palmitic acid ester. Preferred exipients are complexing agents, such as disodium-EDTA and stabilizers, such as a cyclodextrin. The amount and type of excipient added is in accordance with the particular requirements and is generally in the range of from approximately 0.0001 to approximately 90% by weight. A cyclodextrin is composed of several glucose units which have three free hydroxy groups per glucose. The amount of a cyclodextrin used in accordance with one embodiment may preferably range from 0.01-20% by weight, more preferably from 0.1-15% by weight and even more preferably from 1-10% by weight.

Ophthalmic compositions may display pH ranges from 3.5 to 9.0, preferably from 4.5 to 8.0 and most preferably from pH 5.5 to 7.8.

The pH of the composition should preferably be as close to that of the tears as possible. The physiologic pH of tears is approximately 7.4±0.2. Thus, from a comfort, tolerability and safety perspective, this would be the optimal pH of ophthalmic preparations.

Compounds may be included which sooth the eye, reduce surface tension and improve wettability (contact) of an otherwise hydrophobic epithelial corneal surface, approximate the consistency of tears. Such compounds may also enhance the viscosity of the inventive compositions, allowing an inventive formulation to remain in the eye longer thus giving the peptide agent more time to exert its therapeutic activity or undergo absorption to reach the desired target.

Suitable viscosity enhancers in ophthalmic formulations and their concentration ranges used in certain inventive compositions include but are not limited to: (a) Monomeric polyols, such as tyloxapol (0.1-1%), glycerol (0.2-1%), propylene glycol (0.2 to 1%), ethylene glycol (0.2-1%); (b) Polymeric polyols, such as polyethylene glycol (e.g., PEG 300, PEG 400)(0.2-1%); (c) Cellulose derivatives (polymers of the cellulose family), such as hydroxyethylcellulose (0.2-2.5%), hypromellose (0.2 to 2.5%), hydroxypropylmethyl cellulose (0.2-2.5%), methycellulose (0.2-2.5%), carboxymethylcellulose sodium (0.2 to 2.5%), hydroxylpropylcellulose (0.2-2.5%); (d) Dextrans, such as dextran 70 (0.1% when used with another polymeric demulcent agent); (e) Water-soluble proteins such as gelatin (0.01%); (f) Vinyl polymers such as polyvinyl alcohol (0.1-4%), polyvinyl pyrollidine (0.1-4%); (g) Other polyols, such as polysorbate 80 (0.2-1%), povidone (0.1-2%); (h) Carbomers, such as carbomer 934P, carbomer 941, carbomer 940, and carbomer 974P, and (i) Polysaccharides/Glycosaminoglycans, such as hyaluronan (hyaluronic acid/hyaluronate) (0.1-3%), chondroitin sulfate (0.1-3%).

Viscosity describes a material's internal resistance to flow or change in form, when a stress is applied. The viscosity of a material (solution, semi-viscous gel, suspension, oleaginous ointments and ointment gels (viscous gels) is given in poise units. The unit, centipoise (“cp” or the plural “cps”) is equal to 0.01 poise and is most often used in pharmaceutical applications. Compounds used to enhance viscosity are available in various grades such as 15 cps, 100 cps, etc., etc. The grade number refers to the viscosity which results when a fixed percentage aqueous solution of the enhancer is made. Generally, solutions are 1% or 2%; however, they can be as high as 4% with certain enhancers. Viscosity is measured at 20° or 25° C.

A suitable viscosity in an ophthalmic solution is between 25 and 50 centipoises (cps). The actual concentration of an enhancer required to produce that desired viscosity will depend on the grade of the enhancer. For example, if methycellulose 25 cps is used, a 1% solution will create a viscosity of 25 cps. If methycellulose 4000 cps is used, an 0.25% solution provides the desired viscosity. Standard references give tables of viscosities produced by percentage solutions and grades of ingredients.

Preferably, the ophthalmic compositions exhibit a viscosity of >1 to 100,000 centipoises (cps) or greater. Inventive ointment compositions (oleaginous or viscous gels) may have viscosity grades that are greater than 100,000 cps. This is because ophthalmic ointments are intended to be thick when standing to prevent them from flowing away from the intended area of use. Following application and over time, temperatures within the conjunctival sac, or on the surface of the eye, where these ointments are deposited, will cause these ointments to “melt” and begin to flow.

Some compositions have the potential to be degraded by oxidation. Consequently, steps during the manufacture, control and packaging of the composition may include protecting compositions, susceptible to oxidation, by (1) displacing oxygen with nitrogen or a dense inert gas such as argon, (2) adding a reducing agent to minimize oxidative effects, (3) the introduction of a decoy molecule.

Common antioxidant (reducing) agents which may be used in ophthalmic formulations up to a concentration of 0.1% or more are sodium sulfite, sodium thiosulfite, sodium bisulfite, sodium metabisulfite, and thiourea. Sulfites can cause allergic-type reactions in certain people; consequently, patients receiving this type of antioxidant should be questioned about this potential reaction before being treated with an inventive composition containing the antioxidant. Other useful antioxidants compatible with the inventive compositions are ascorbic acid, EDTA/disodium edetate, acetic acid, citric acid, glutathione and acetylcysteine. These agents may also be regarded as stabilizers.

A decoy molecule or an oxygen sequestering protective agent may be added as stabilizers to composition to minimize oxidative effects on the inventive formulation. The molecular decoy must have at least the same capability of being oxidized as the composition. One such decoy, for a composition containing methionine is the amino acid, methionine, itself. Free methionine added to an inventive composition containing the amino acid methionine would compete for oxygen in the process of being oxidized to methionyl sulfoxide. A free oxygen-consuming agent is one that prevents other oxygen-reactive amino acids in the inventive composition/peptide from being oxidized. For the purposes of certain inventive compositions but not limited to such, a free oxygen-consuming agent is methionine.

Ophthalmic ointments tend to keep an active agent in contact with the eye longer than suspensions and certainly solutions. Most ointments, tend to blur vision, as they are not removed easily by the tear fluid. Thus, ointments are generally used at night as adjunctive therapy to eye drops used during the day.

Oleaginous ointment bases of inventive compositions are mixtures of mineral oil, petrolatum and lanolin all have a melting point close to body temperature. In the case of the inventive compounds, the compositions may include mineral oil, petrolatum or lanolin. According to one embodiment preferred compositions would include a combination of petrolatum, mineral oil and lanolin. The most preferred composition is an ointment combination containing white petrolatum, mineral oil and lanolin (anhydrous).

Such compositions are prepared in standard ways, for example by mixing the active ingredient(s) with the corresponding excipients and/or additives to form corresponding ophthalmic compositions.

For example, where the composition is administered in the form of eye drops, the active ingredient(s) are dissolved, for example, in a carrier. The solution is, where appropriate, adjusted and/or buffered to the desired pH and, where appropriate, a stabilizer, a solubilizer or a tonicity enhancing agent is added. Where appropriate, preservatives and/or other excipients are added to the composition.

The composition may, for example, be in the form of a solution, a suspension, an ointment, a gel or a foam, inter alia, particularly for topical and sub-conjunctival administration. Preferably, the composition is in the form of a solution or gel.

In some embodiments, the composition is not a nanocrystalline formulation. In particular, in some embodiments, the composition does not comprise a double-soluble macromolecule (e.g. a surfactant) and a single-soluble macromolecule (e.g. a starch or cellulose-based compound) which interact to encapsulate the RTK inhibitor. The terms “nanocrystalline formulation”, “double-soluble macromolecule” and a “single-soluble macromolecule” may be as defined and exemplified in CN110664757A.

In additional to a RTK inhibitor, an eye drop solution may comprise one or more or all components selected from chloramphenicol (5 mg per 1 ml solution), boric acid, sodium borate, sterile water, and benzalkonium chloride.

In additional to a RTK inhibitor, a 1 ml eye drop suspension may comprise one or more or all components selected from Tobramycin (3 mg), dexamethasone (1 mg), benzalkonium chloride, tyloxapol, hydroxyethylcellulose, disodium EDTA, sodium sulphate anhydrate, sodium chloride, sulphuric acid and/or sodium hydroxide for pH-adjustment, and purified water.

In additional to a RTK inhibitor, a 1 g eye ointment may comprise one or more or all components selected from chloramphenicol (10 mg), liquid paraffin and white soft paraffin.

In additional to a RTK inhibitor, a 1 ml sustained release eye drop gel may comprise one or more or all components selected from Timololmaleate (1.37 mg), benzalkonium chloride (0.05 mg), sorbitol, polyvinylalcohol, carbomer, sodiumacetate trihydrate, lysinemonohydrate and sterile water.

In additional to a RTK inhibitor, an eye drop foam may comprise a foaming agent, preferably hydroxypropyl methylcellulose (hypromellose) or albumin.

Nanotechnology may be used in the management of ocular diseases by providing controlled release, ensuring low eye irritation, improving drug bioavailability or enhancing ocular tissue compatibility (see Weng et al. Acta Pharmaceutica Sinica B 2017; 7(3): 281-291). Various nanosystems have been designed to deliver their payloads into both anterior and posterior segments of the eye. These nanosystems are mainly made from natural or synthetic polymeric materials. Many colloidal systems such as micelles, liposomes, niosomes, dendrimers, in situ hydrogels, and cyclodextrins are of this type. Other forms, including nanoparticles, implants and nanoparticle-contained contact lens, films, as well as other delivery systems, have also been used.

In yet a further embodiment, therefore, the RTK inhibitor (preferably dasatinib) is provided in the form of a nanotechnology-based or nanoparticle-based ocular delivery system, preferably selected from the group consisting of nanospheres, nanocapsules, liposomes, hydrogels, dendrimers, nanoparticles and nanomicelles.

The RTK inhibitor (preferably dasatinib) may also be provided in the form of a nanoparticle-loaded contact lens. Such lenses form a further aspect of the invention.

Preferably, the pharmaceutical composition is packaged in a form which is adapted to dispense the pharmaceutical composition into the eye or onto the cornea. Preferably, the form is adapted to dispense one or more eye drops. The composition is preferably packaged in the form of an eye drop bottle, eye drop flask, eye drop dispenser, eye dropper, or an ophthalmic ointment tube.

Eye-drops are a (generally) saline-based solution which is administrated to an eye for lubrication and/or delivery of medication. Typically, eye-drops are sold contained in an eye drop bottle. The eye drop bottle is often designed with a nozzle that allows eye-drops to be dispensed therefrom upon squeezing of the eye drop bottle.

Ophthalmic ointment tubes are typically small tubes holding approximately 1-5 grams of ointment, preferably 3.5 grams, and fitted with narrow gauge tips which permit the extrusion of narrow bands of ointment measured in inches or fractions thereof for dosing purposes.

The composition may be packaged in the form of a single-use dispenser.

The invention also provides a dispensing device adapted to deliver liquid drops (of the composition of the invention), the dispensing device containing a pharmaceutical composition of the invention, the dispensing device preferably being in the form of an eye drop bottle, eye drop flask, eye drop dispenser, or eye dropper, optionally together with instructions for use.

In yet another embodiment, the invention provides a method of diagnosing OPDKD in a subject, the method comprising the steps:

(a) detecting, from a biological sample obtained from the subject, whether the subject's PDGFRβ gene encodes the mutation N666Y or S548Y; and

(b) diagnosing the subject with OPDKD if the presence of the N666Y and/or S548Y mutation in the biological sample is detected.

The biological sample may be a sample which comprises the PDGFRβ protein or nucleic acid (DNA or RNA) which encodes the PDGFRβ protein. For example, the biological sample may be a tissue sample or one or more cells from the subject. The biological sample may, for example, be a sample of blood, plasma or serum.

In some embodiments, the method comprises the step (prior to Step (a)) of obtaining the biological sample from the subject.

The presence of the mutation N666Y or S548Y in the PDGFRβ gene may be detected by any suitable means, e.g. using an antibody which is specific for the N666Y or S548Y mutation or DNA sequencing of the nucleic acid (DNA or RNA) which encodes the subject's PDGFRβ protein. The mutation in the PDGFRβ gene may be homozygous or heterozygous.

The method of diagnosing OPDKD may also comprise the step of:

(c) administering an effective amount of an RTK inhibitor to the diagnosed patient.

Preferably, the method of diagnosing is an in vitro or ex vivo method.

In yet other embodiments, the invention provides a pharmaceutical composition comprising an RTK inhibitor for use in treating or preventing OPDKD or WCS in a subject. In yet other embodiments, the invention provides a method of treating or preventing OPDKD or WCS in a subject, the method comprising administering an effective amount of a composition comprising an RTK inhibitor to the subject. In yet other embodiments, the invention provides the use of an RTK inhibitor in the manufacture of a medicament for treating or preventing OPDKD or WCS in a subject.

Preferably, the OPDKD is associated with or caused by the aberrant expression of a receptor tyrosine kinase (RTK) in the subject, more preferably wherein the RTK is PDGFRβ. In some embodiments, the OPDKD is due to one or more mutations in the PDGFRβ gene, for example, a mutation which leads to increased activation of the PDGFRβ protein. In some embodiments, the mutation is N666Y in the PDGFRβ gene.

Preferably, the WCS is associated with or caused by the aberrant expression of a receptor tyrosine kinase (RTK) in the subject, more preferably wherein the RTK is DDR2. In some embodiments, the WCS is due to one or more mutations in the DDR2 gene, for example, a mutation which leads to increased activation of the DDR2 protein. In some embodiments, the mutation in the DDR2 gene encodes Y740C and/or L610P.

In some embodiments, the pharmaceutical composition is in the form of a topical composition, e.g. one which is suitable for application to a subject's fingers and/or toes. This is suitable for the treatment of keloids/chronic ulcers on fingers, toes and elsewhere on the body.

The disclosure of each reference set forth herein is specifically incorporated herein by reference in its entirety.

### BRIEF DESCRIPTION OF THE FIGURES

FIG. 1. Figure of the pedigree of the original OPDKD family showing the results from haplotype analysis. While two healthy family members share the same gene string, the PDGFRβ mutation has occurred in the index patient (19*) proving that it is a de novo mutation.

FIG. 2. Results from ELISA analysis of healthy, OPDKD (N666Y) and Penttinen fibroblasts (N666S) showing significantly increased levels of phosphorylated PDGFRβ in OPDKD fibroblasts cultured at 32° C. for 6 hours. This shows that the N666Y mutation is activating and temperature sensitive. In contrast, the N666S mutation has comparable levels of phosphorylated PDGFRβ at reduced temperatures.

FIG. 3. To the left, a dose-dependent effect of imatinib was seen leading do normalization of levels of phosphorylated PDGFRβ in patent fibroblasts treated at high doses of imatinib (1 μM). To the right, the effect of downstream signaling partner AKT was seen. Increased phosphorylation of both Ser473 and Thr308 AKT was seen in patient fibroblasts, with increasing levels at reduced temperatures. Similar levels of unphosphorylated AKT were seen. Normalization was seen after treatment with 0.1 μM imatinib.

FIG. 4. Pictures of the eye of an OPDKD patient treated for a year with imatinib; no effect of the drug was seen.

FIG. 5. Control fibroblasts had no detectable phosphorylated DDR2. DDR2 Y740 fibroblasts had higher levels at 32° C. than at 37° C. DDR2L610P fibroblasts were not temperature-sensitive with similar levels of phosphorylated DDR2 at both temperatures. Treatment with 0.1 μM dasatinib for 6 hours lead to greatly reduced levels of phosphorylated DDR2.* loading control (GAPDH).

FIG. 6. ELISA analysis of levels of phosphorylated PDGFRβ in Hela cells transduced with the S548Y variant.

FIGS. 7A-7D. Hela cells transduced with the specified PDGFRβ mutations and treated with different RTK inhibitors.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Identification of Mutation in PDGFRβ in OPDKD Patients

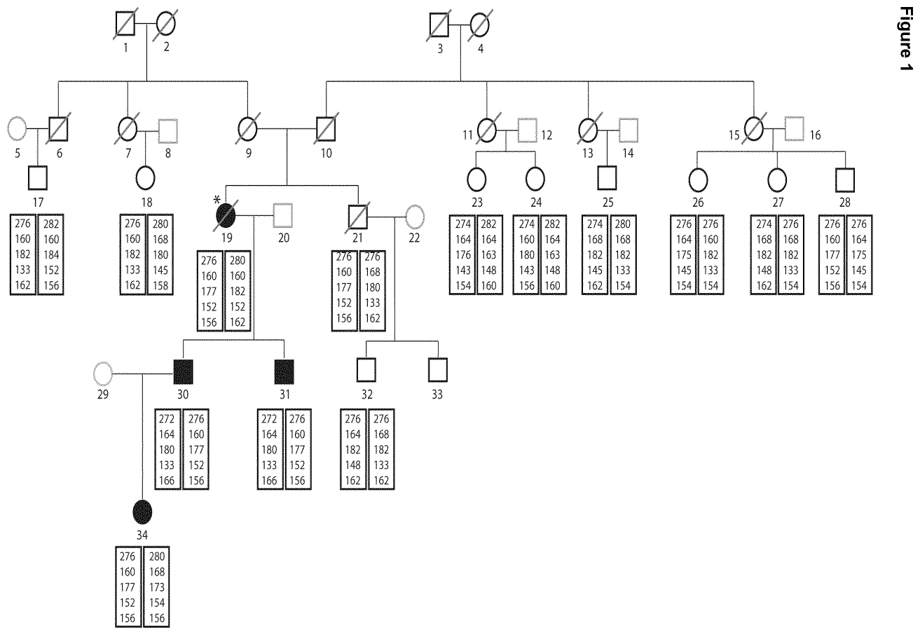

DNA from affected and unaffected family members was subjected to exome sequencing using NimbleGen v3 exome capture on Illumina HiSeq. Analysis of the exome sequencing results (FIG. 1) showed a variant which was shared by all affected individuals and not present in any of the unaffected family members: PDGFRβ:NM_002609.3:exon14:c.A1996T:p. N666Y.

The PDGFRβ missense variant changes an amino acid located in the RTK class III signature motif of PDGFRβ, an essential part of the auto-inhibitory domain. This motif is highly conserved; variants in this codon have previously been associated with Penttinen syndrome and infantile myofibromatosis^{3; 5-7}.

Haplotype analysis with microsatellite markers around the PDGFRβ locus was then performed. This analysis around the PDGFRβlocus in the extended family demonstrated that the variant occurred as a de novo mutation in individual I-1.

### Example 2: Identification of Mutation N666Y as Being Temperature Sensitive

Fibroblasts from affected individuals and healthy controls were obtained. Transgenic HeLa cells were transduced with a murine retroviral vector containing the PDGFRβ (NM_002609.3) c.wt, c. 1996A>T (OPDKD) and c.1997A>G (a different activating mutation associated with severe Penttinen syndrome) variants.

In OPDKD, the affected parts of the body (corneas and skin, particular fingers and toes) are all exposed to variable temperatures. ⁸ At a typical room temperature (20-22° C.) corneal temperatures lie around 32-34° and fall to around 30° C. as air temperatures reaches 0° C. ⁹ We studied the effect of this physiological temperature difference on PDGFRβ auto-phosphorylation and signaling in OPDKD. Cells were either kept at 37° ° C. or incubated overnight at 32° C. Cells were then left untreated or treated with 0.1 μM imatinib (STI571, Selleckchem) for 6 hours. For ELISA analysis of phosphorylated PDGFRβ, a DuoSet® IC Phospho-PDGFRβ kit (R&D systems) was used. Cell lysates were also subjected to immunoblot analysis with primary antibodies against downstream signaling partners: phospho-Ser473-AKT, phospho-Thr308-AKT, phospho-Tyr70-STAT1, STAT1, phospho-Tyr783-PLCγ1, PLCγ1, phospho-Thr202/Tyr204-MAPK3/ERK1 and MAPK3/ERK1 (all from Cell Signaling Technology).

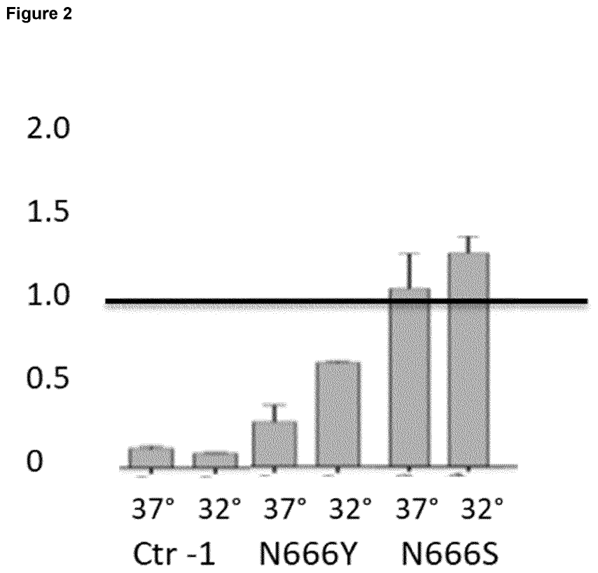

At 37° C. increased levels of phosphorylated PDGFRβ and downstream proteins P-AKT and phospholipase Cγ (PLCγ1) were present in OPDKD cells compared to controls. However, both auto-phosphorylation and signaling were greatly increased at 32° C. (FIGS. 2A and B left). This indicates that the N666Y substitution diminishes auto-inhibition of PDGFRβ and that the process is temperature sensitive. In transduced HeLa cells similar findings were made.

In contrast, in fibroblasts and transduced Hela cells with the N666S variant, levels of phosphorylated PDGFRβ were not affected by temperature.

### Example 3: Treatment of Fibroblasts With Imatinib Normalised P-PDGFRβ Levels

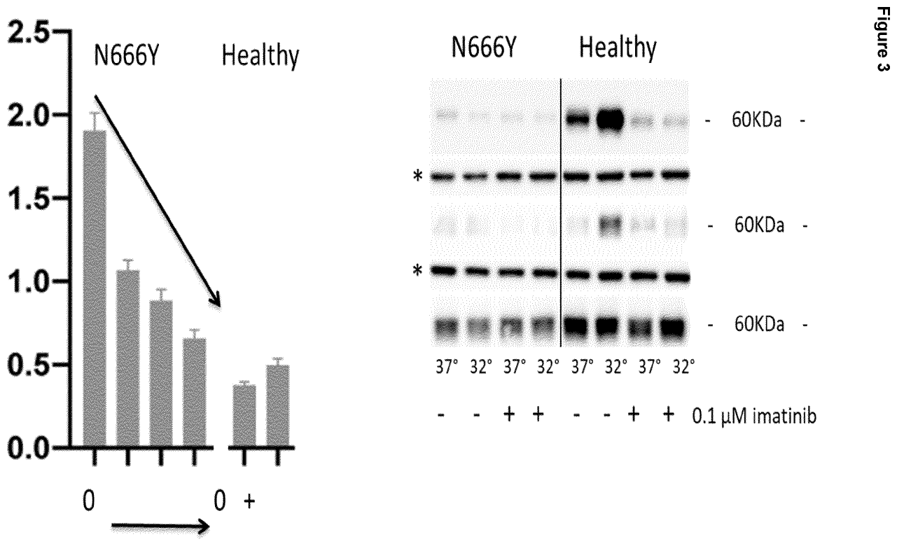

Cells were then left untreated or treated with 0.1 μM imatinib (STI571, Selleckchem) for 6 hours. OPDKD fibroblasts seemed less sensitive to imatinib than Penttinnen fibroblasts but a clear, dose dependent effect was seen in levels of P-PDGFRβ and downstream proteins (FIG. 3).

### Example 4: Oral Administration of Imatinib was Not Efficacious in the Eye

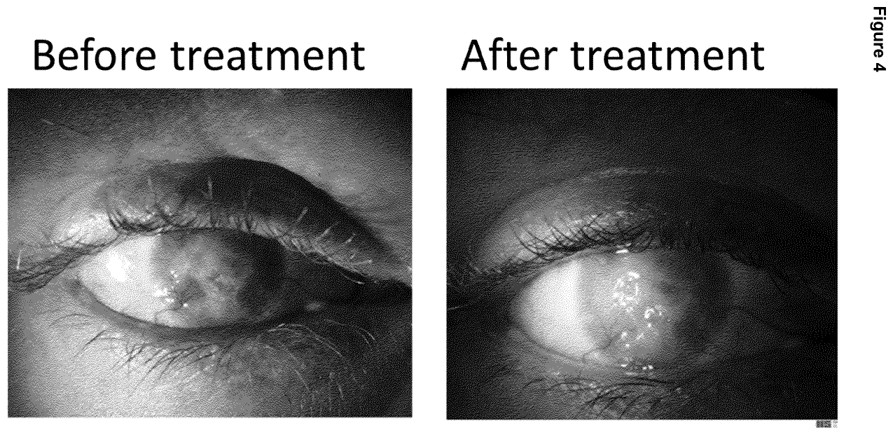

One OPDKD patient was treated for almost one year with systemic imatinib, for 6 months using the maximal recommended dose of 800 mg, without effect (FIG. 4).

### Example 5: Identification of DDR2 Mutation Y740C as Being Temperature Sensitive

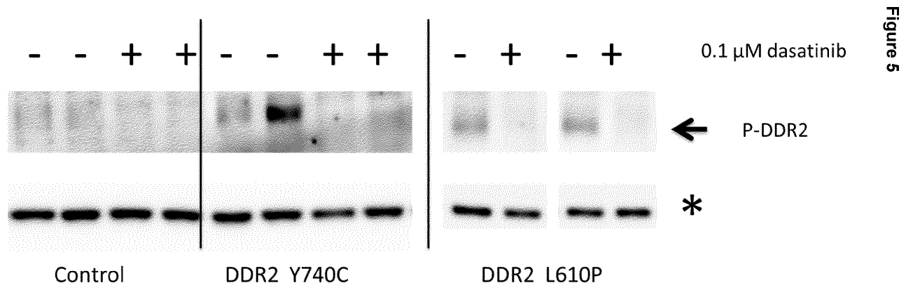

We noticed that patients with Warburg-Cinotti syndrome had a more severe ocular phenotype in comparison with their other clinical problems. This pattern resembled OPDKD patients and this lead us to examine the effect of the physiological temperature difference on DDR2 auto-phosphorylation in Warburg-Cinotti syndrome. We found that after long (hours) exposure to reduced temperatures (32° C.) increased levels of activated (phosphorylated) DDR2 were present in fibroblasts with the Y740C mutation (FIG. 5). In contrast, the L610P mutation was not temperature sensitive.

### Example 6. ELISA Analysis of Levels of Phosphorylated PDGFRβ in Hela Cells Transduced With the S548Y Variant

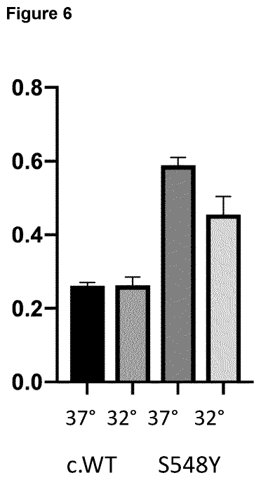

ELISA analysis of levels of phosphorylated PDGFRβ in Hela cells transduced with the S548Y variant showed increased levels compared to control (FIG. 6). This was not further elevated at reduced temperatures. This shows that the mutation is activating but not temperature-sensitive.

### Example 7: Eye-Drop Formulation

An eye-drop formulation is made containing dasatinib, boric acid, sodium borate, sterile water and benzalkonium chloride, using standard methods.

### Example 8: Administration of Eye-Drop Formulation

The eye-drop formulation of Example 7 is administered directly onto the corneas of patients, at a dosage of 1-8 drops per day.

### Example 9: Effects of Different -Inib Drugs on Hela Cells

Hela cells transduced with the following PDGFRβ mutations (WT, N666S, P584R, R561C or W566R corresponding to the following mutations: normal PDGFRβ, Asn666Ser (Penttinen), Pro584Arg, Trp566Arg (both Kosaki) and Arg561Cys (infantile myofibromatosis) were cultured under standard conditions. They were then transferred to serum-free media and left untreated or treated with IC50 (as provided from the manufacturer Selleckchem) or 10x this concentration for 6 or 24 hours.

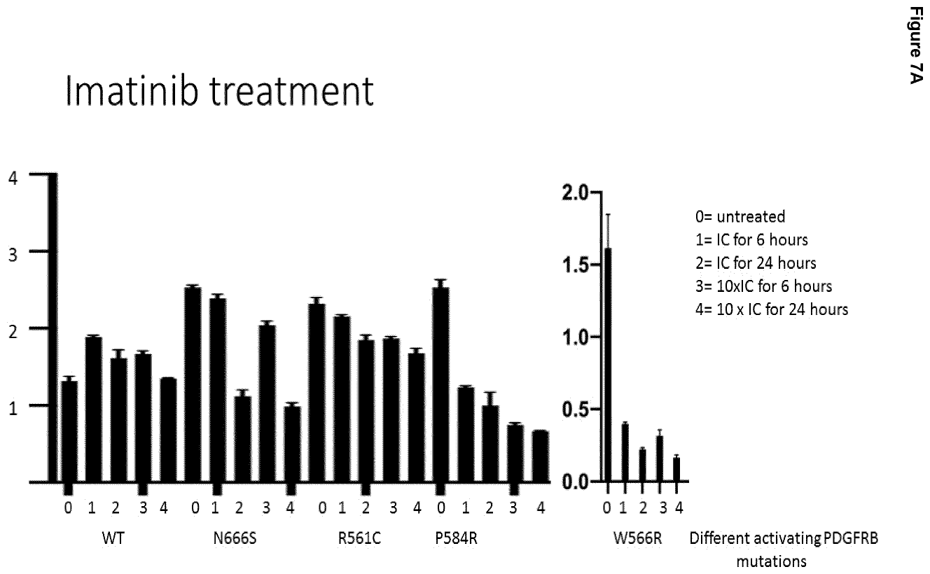

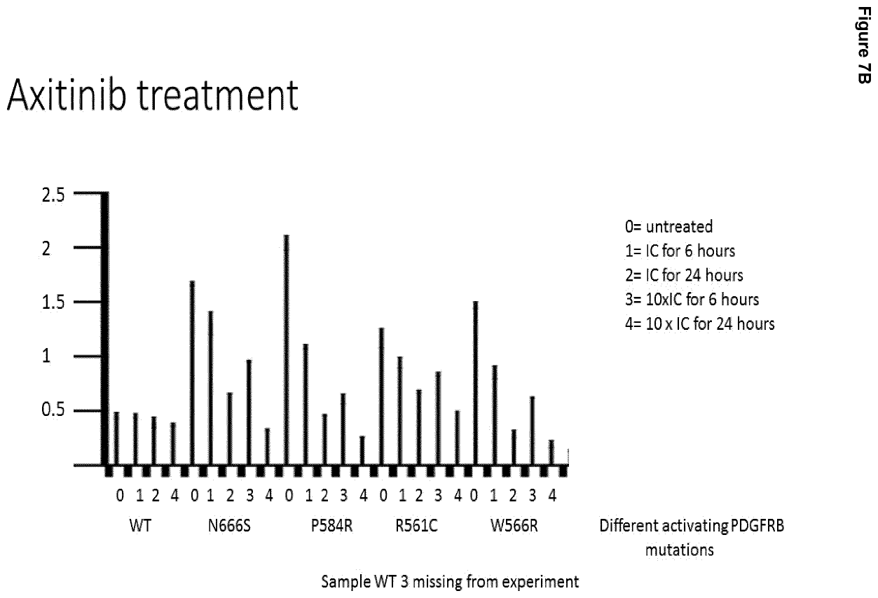

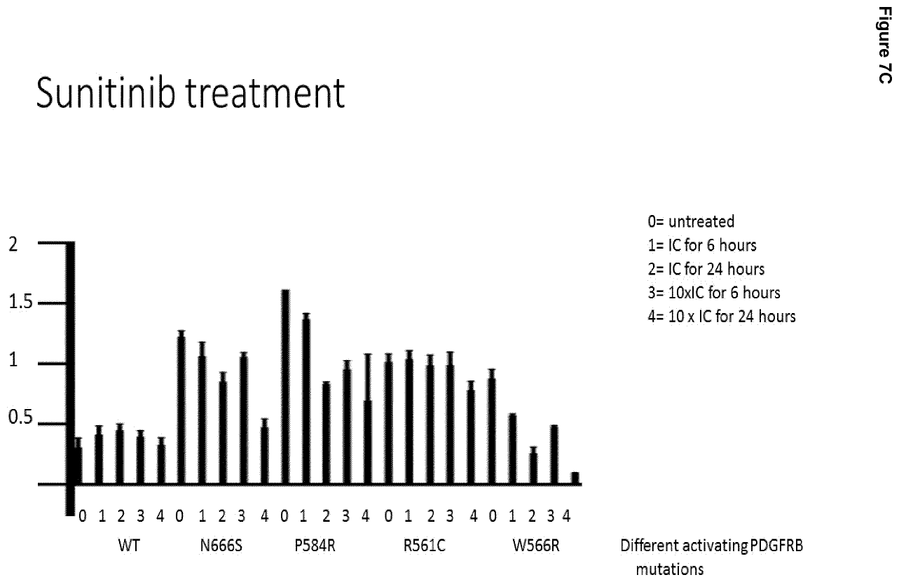

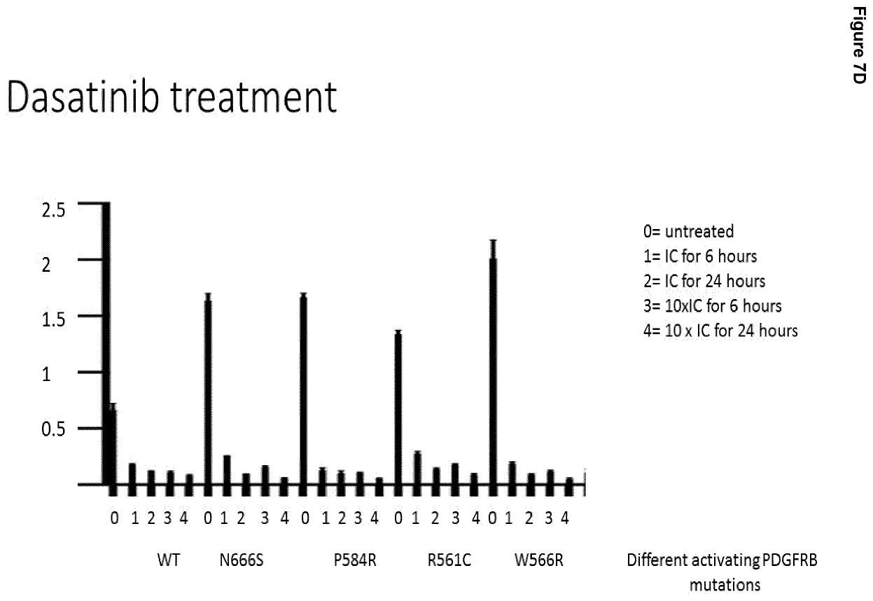

The results are shown in FIGS. 7A-7D. For the different mutations, various sensitivity to the different RTK inhibitors are seen. However, for dasatinib a strong effect was seen at all time-points, all concentrations and for all of the tested mutations.

### REFERENCES

1. Abarca, H., Mellgren, A. E., Trubnykova, M., Haugen, O. H., Hovding, G., Tveit, K. S., Houge, G., Bredrup, C., and Hennekam, R. C. (2014). Ocular pterygium—digital keloid dysplasia. Am J Med Genet A 164A, 2901-2907.

2. Haugen, O. H., and Bertelsen, T. (1998). A new hereditary conjunctivo-corneal dystrophy associated with dermal keloid formation. Report of a family. Acta Ophthalmol Scand 76, 461-465.

3. Pond, D., Arts, F. A., Mendelsohn, N. J., Demoulin, J. B., Scharer, G., and Messinger, Y. (2017). A patient with germ-line gain-of-function PDGFRB p.N666H mutation and marked clinical response to imatinib. Genet Med.

4. Mudry, P., Slaby, O., Neradil, J., Soukalova, J., Melicharkova, K., Rohleder, O., Jezova, M., Seehofnerova, A., Michu, E., Veselska, R., et al. (2017). Case report: rapid and durable response to PDGFR targeted therapy in a child with refractory multiple infantile myofibromatosis and a heterozygous germline mutation of the PDGFRB gene. BMC Cancer 17, 119.

5. Chen, H., Marsiglia, W. M., Cho, M. K., Huang, Z., Deng, J., Blais, S. P., Gai, W., Bhattacharya, S., Neubert, T. A., Traaseth, N. J., et al. (2017). Elucidation of a four-site allosteric network in fibroblast growth factor receptor tyrosine kinases. Elife 6.

6. Bredrup, C., Stokowy, T., McGaughran, J., Lee, S., Sapkota, D., Cristea, I., Xu, L., Tveit, K. S., Hovding, G., Steen, V. M., et al. (2018). A tyrosine kinase-activating variant Asn666Ser in PDGFRB causes a progeria-like condition in the severe end of Penttinen syndrome. Eur J Hum Genet.

7. Cheung, Y. H., Gayden, T., Campeau, P. M., LeDuc, C. A., Russo, D., Nguyen, V. H., Guo, J., Qi, M., Guan, Y., Albrecht, S., et al. (2013). A recurrent PDGFRB mutation causes familial infantile myofibromatosis. Am J Hum Genet 92, 996-1000.

8. Girardin, F., Orgul, S., Erb, C., and Flammer, J. (1999). Relationship between corneal temperature and finger temperature. Arch Ophthalmol 117, 166-169.

9. Webb, P. (1992). Temperatures of skin, subcutaneous tissue, muscle and core in resting men in cold, comfortable and hot conditions. Eur J Appl Physiol Occup Physiol 64, 471-476.

### SEQUENCES

Any Sequence Listing filed with this patent application is fully incorporated herein as part of the description.

SEQ ID NO: 1
Protein name: Wild-type PDGFRß PDGFRB_NP_002600:
Origin: Human
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGE
YFCTHNDSRGLETDERKRLYIFVPDPTVGFLPNDAEELFIFLTEITEITIPCRVTDPQLVVTLHEKKGDVALPVPYDHQRGFSGIFEDRSYICKTTI
GDREVDSDAYYVYRLQVSSINVSVNAVQTVVRQGENITLMCIVIGNEVVNFEWTYPRKESGRLVEPVTDFLLDMPYHIRSILHIPSAELEDSGTYTC
NVTESVNDHQDEKAINITVVESGYVRLLGEVGTLQFAELHRSRTLQVVFEAYPPPTVLWFKDNRTLGDSSAGEIALSTRNVSETRYVSELTLVRVKV
AEAGHYTMRAFHEDAEVQLSFQLQINVPVRVLELSESHPDSGEQTVRCRGRGMPQPNIIWSACRDLKRCPRELPPTLLGNSSEEESQLETNVTYWEE
EQEFEVVSTLRLQHVDRPLSVRCTLRNAVGODTQEVIVVPHSLPFKVVVISAILALVVLTIISLIILIMLWQKKPRYEIRWKVIESVSSDGHEYIYV
DPMQLPYDSTWELPRDQLVLGRTLGSGAFGQVVEATAHGLSHSQATMKVAVKMLKSTARSSEKQALMSELKIMSHLGPHLNVVNLLGACTKGGPIYI
ITEYCRYGDLVDYLHRNKHTFLQHHSDKRRPPSAELYSNALPVGLPLPSHVSLTGESDGGYMDMSKDESVDYVPMLDMKGDVKYADIESSNYMAPYD
NYVPSAPERTCRATLINESPVLSYMDLVGFSYQVANGMEFLASKNCVHRDLAARNVLICEGKLVKICDFGLARDIMRDSNYISKGSTFLPLKWMAPE
SIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKY
QQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQ
DEPEPEPQLELQVEPEPELEQLPDSGCPAPRAEAEDSFL
SEQ ID NO: 2
Protein name: Wild-type PDGFRß (PDGFRB_NM_002609.4:)
Origin: Human
AGCAGCCAGCAGTGACTGCCCGCCCTATCTGGGACCCAGGATCGCTCTGTGAGCAACTTGGAGCCAGAGAGGAGATCAACAAGGAGGAGGA
GAGAGCCGGCCCCTCAGCCCTGCTGCCCAGCAGCAGCCTGTGCTCGCCCTGCCCAACGCAGACAGCCAGACCCAGGGCGGCCCCTCTGGCGG
CTCTGCTCCTCCCGAAGGATGCTTGGGGAGTGAGGCGAAGCTGGGCCGCTCCTCTCCCCTACAGCAGCCCCCTTCCTCCATCCCTCTGTTCTCCT
GAGCCTTCAGGAGCCTGCACCAGTCCTGCCTGTCCTTCTACTCAGCTGTTACCCACTCTGGGACCAGCAGTCTTTCTGATAACTGGGAGAGGGC
AGTAAGGAGGACTTCCTGGAGGGGGTGACTGTCCAGAGCCTGGAACTGTGCCCACACCAGAAGCCATCAGCAGCAAGGACACCATGCGGCTT
CCGGGTGCGATGCCAGCTCTGGCCCTCAAAGGCGAGCTGCTGTTGCTGTCTCTCCTGTTACTTCTGGAACCACAGATCTCTCAGGGCCTGGTCG
TCACACCCCCGGGGCCAGAGCTTGTCCTCAATGTCTCCAGCACCTTCGTTCTGACCTGCTCGGGTTCAGCTCCGGTGGTGTGGGAACGGATGTC
CCAGGAGCCCCCACAGGAAATGGCCAAGGCCCAGGATGGCACCTTCTCCAGCGTGCTCACACTGACCAACCTCACTGGGCTAGACACGGGAG
AATACTTTTGCACCCACAATGACTCCCGTGGACTGGAGACCGATGAGCGGAAACGGCTCTACATCTTTGTGCCAGATCCCACCGTGGGCTTCCT
CCCTAATGATGCCGAGGAACTATTCATCTTTCTCACGGAAATAACTGAGATCACCATTCCATGCCGAGTAACAGACCCACAGCTGGTGGTGACA
CTGCACGAGAAGAAAGGGGACGTTGCACTGCCTGTCCCCTATGATCACCAACGTGGCTTTTCTGGTATCTTTGAGGACAGAAGCTACATCTGCA
AAACCACCATTGGGGACAGGGAGGTGGATTCTGATGCCTACTATGTCTACAGACTCCAGGTGTCATCCATCAACGTCTCTGTGAACGCAGTGC
AGACTGTGGTCCGCCAGGGTGAGAACATCACCCTCATGTGCATTGTGATCGGGAATGAGGTGGTCAACTTCGAGTGGACATACCCCCGCAAA
GAAAGTGGGCGGCTGGTGGAGCCGGTGACTGACTTCCTCTTGGATATGCCTTACCACATCCGCTCCATCCTGCACATCCCCAGTGCCGAGTTA
GAAGACTCGGGGACCTACACCTGCAATGTGACGGAGAGTGTGAATGACCATCAGGATGAAAAGGCCATCAACATCACCGTGGTTGAGAGCGG
CTACGTGCGGCTCCTGGGAGAGGTGGGCACACTACAATTTGCTGAGCTGCATCGGAGCCGGACACTGCAGGTAGTGTTCGAGGCCTACCCAC
CGCCCACTGTCCTGTGGTTCAAAGACAACCGCACCCTGGGCGACTCCAGCGCTGGCGAAATCGCCCTGTCCACGCGCAACGTGTCGGAGACCC
GGTATGTGTCAGAGCTGACACTGGTTCGCGTGAAGGTGGCAGAGGCTGGCCACTACACCATGCGGGCCTTCCATGAGGATGCTGAGGTCCAG
CTCTCCTTCCAGCTACAGATCAATGTCCCTGTCCGAGTGCTGGAGCTAAGTGAGAGCCACCCTGACAGTGGGGAACAGACAGTCCGCTGTCGT
GGCCGGGGCATGCCCCAGCCGAACATCATCTGGTCTGCCTGCAGAGACCTCAAAAGGTGTCCACGTGAGCTGCCGCCCACGCTGCTGGGGAA
CAGTTCCGAAGAGGAGAGCCAGCTGGAGACTAACGTGACGTACTGGGAGGAGGAGCAGGAGTTTGAGGTGGTGAGCACACTGCGTCTGCAG
CACGTGGATCGGCCACTGTCGGTGCGCTGCACGCTGCGCAACGCTGTGGGCCAGGACACGCAGGAGGTCATCGTGGTGCCACACTCCTTGCC
CTTTAAGGTGGTGGTGATCTCAGCCATCCTGGCCCTGGTGGTGCTCACCATCATCTCCCTTATCATCCTCATCATGCTTTGGCAGAAGAAGCCAC
GTTACGAGATCCGATGGAAGGTGATTGAGTCTGTGAGCTCTGACGGCCATGAGTACATCTACGTGGACCCCATGCAGCTGCCCTATGACTCCA
CGTGGGAGCTGCCGCGGGACCAGCTTGTGCTGGGACGCACCCTCGGCTCTGGGGCCTTTGGGCAGGTGGTGGAGGCCACGGCTCATGGCCT
GAGCCATTCTCAGGCCACGATGAAAGTGGCCGTCAAGATGCTTAAATCCACAGCCCGCAGCAGTGAGAAGCAAGCCCTTATGTCGGAGCTGA
AGATCATGAGTCACCTTGGGCCCCACCTGAACGTGGTCAACCTGTTGGGGGCCTGCACCAAAGGAGGACCCATCTATATCATCACTGAGTACT
GCCGCTACGGAGACCTGGTGGACTACCTGCACCGCAACAAACACACCTTCCTGCAGCACCACTCCGACAAGCGCCGCCCGCCCAGCGCGGAG
CTCTACAGCAATGCTCTGCCCGTTGGGCTCCCCCTGCCCAGCCATGTGTCCTTGACCGGGGAGAGCGACGGTGGCTACATGGACATGAGCAAG
GACGAGTCGGTGGACTATGTGCCCATGCTGGACATGAAAGGAGACGTCAAATATGCAGACATCGAGTCCTCCAACTACATGGCCCCTTACGAT
AACTACGTTCCCTCTGCCCCTGAGAGGACCTGCCGAGCAACTTTGATCAACGAGTCTCCAGTGCTAAGCTACATGGACCTCGTGGGCTTCAGCT
ACCAGGTGGCCAATGGCATGGAGTTTCTGGCCTCCAAGAACTGCGTCCACAGAGACCTGGCGGCTAGGAACGTGCTCATCTGTGAAGGCAAG
CTGGTCAAGATCTGTGACTTTGGCCTGGCTCGAGACATCATGCGGGACTCGAATTACATCTCCAAAGGCAGCACCTTTTTGCCTTTAAAGTGGA
TGGCTCCGGAGAGCATCTTCAACAGCCTCTACACCACCCTGAGCGACGTGTGGTCCTTCGGGATCCTGCTCTGGGAGATCTTCACCTTGGGTG
GCACCCCTTACCCAGAGCTGCCCATGAACGAGCAGTTCTACAATGCCATCAAACGGGGTTACCGCATGGCCCAGCCTGCCCATGCCTCCGACG
AGATCTATGAGATCATGCAGAAGTGCTGGGAAGAGAAGTTTGAGATTCGGCCCCCCTTCTCCCAGCTGGTGCTGCTTCTCGAGAGACTGTTGG
GCGAAGGTTACAAAAAGAAGTACCAGCAGGTGGATGAGGAGTTTCTGAGGAGTGACCACCCAGCCATCCTTCGGTCCCAGGCCCGCTTGCCT
GGGTTCCATGGCCTCCGATCTCCCCTGGACACCAGCTCCGTCCTCTATACTGCCGTGCAGCCCAATGAGGGTGACAACGACTATATCATCCCCC
TGCCTGACCCCAAACCCGAGGTTGCTGACGAGGGCCCACTGGAGGGTTCCCCCAGCCTAGCCAGCTCCACCCTGAATGAAGTCAACACCTCCT
CAACCATCTCCTGTGACAGCCCCCTGGAGCCCCAGGACGAACCAGAGCCAGAGCCCCAGCTTGAGCTCCAGGTGGAGCCGGAGCCAGAGCTG
GAACAGTTGCCGGATTCGGGGTGCCCTGCGCCTCGGGCGGAAGCAGAGGATAGCTTCCTGTAGGGGGCTGGCCCCTACCCTGCCCTGCCTGA
AGCTCCCCCCCTGCCAGCACCCAGCATCTCCTGGCCTGGCCTGACCGGGCTTCCTGTCAGCCAGGCTGCCCTTATCAGCTGTCCCCTTCTGGAA
GCTTTCTGCTCCTGACGTGTTGTGCCCCAAACCCTGGGGCTGGCTTAGGAGGCAAGAAAACTGCAGGGGCCGTGACCAGCCCTCTGCCTCCAG
GGAGGCCAACTGACTCTGAGCCAGGGTTCCCCCAGGGAACTCAGTTTTCCCATATGTAAAATGGGAAAGTTAGGCTTGATGACCCAGAATCTA
GGATTCTCTCCCTGGCTGACAGGTGGGGAGACCGAATCCCTCCCTGGGAAGATTCTTGGAGTTACTGAGGTGGTAAATTAACTTTTTTCTGTTC
AGCCAGCTACCCCTCAAGGAATCATAGCTCTCTCCTCGCACTTTTATCCACCCAGGAGCTAGGGAAGAGACCCTAGCCTCCCTGGCTGCTGGCT
GAGCTAGGGCCTAGCCTTGAGCAGTGTTGCCTCATCCAGAAGAAAGCCAGTCTCCTCCCTATGATGCCAGTCCCTGCGTTCCCTGGCCCGAGCT
GGTCTGGGGCCATTAGGCAGCCTAATTAATGCTGGAGGCTGAGCCAAGTACAGGACACCCCCAGCCTGCAGCCCTTGCCCAGGGCACTTGGA
GCACACGCAGCCATAGCAAGTGCCTGTGTCCCTGTCCTTCAGGCCCATCAGTCCTGGGGCTTTTTCTTTATCACCCTCAGTCTTAATCCATCCAC
CAGAGTCTAGAAGGCCAGACGGGCCCCGCATCTGTGATGAGAATGTAAATGTGCCAGTGTGGAGTGGCCACGTGTGTGTGCCAGTATATGGC
CCTGGCTCTGCATTGGACCTGCTATGAGGCTTTGGAGGAATCCCTCACCCTCTCTGGGCCTCAGTTTCCCCTTCAAAAAATGAATAAGTCGGACT
TATTAACTCTGAGTGCCTTGCCAGCACTAACATTCTAGAGTATTCCAGGTGGTTGCACATTTGTCCAGATGAAGCAAGGCCATATACCCTAAACT
TCCATCCTGGGGGTCAGCTGGGCTCCTGGGAGATTCCAGATCACACATCACACTCTGGGGACTCAGGAACCATGCCCCTTCCCCAGGCCCCCA
GCAAGTCTCAAGAACACAGCTGCACAGGCCTTGACTTAGAGTGACAGCCGGTGTCCTGGAAAGCCCCCAGCAGCTGCCCCAGGGACATGGGA
AGACCACGGGACCTCTTTCACTACCCACGATGACCTCCGGGGGTATCCTGGGCAAAAGGGACAAAGAGGGCAAATGAGATCACCTCCTGCAG
CCCACCACTCCAGCACCTGTGCCGAGGTCTGCGTCGAAGACAGAATGGACAGTGAGGACAGTTATGTCTTGTAAAAGACAAGAAGCTTCAGAT
GGGTACCCCAAGAAGGATGTGAGAGGTGGGCGCTTTGGAGGTTTGCCCCTCACCCACCAGCTGCCCCATCCCTGAGGCAGCGCTCCATGGGG
GTATGGTTTTGTCACTGCCCAGACCTAGCAGTGACATCTCATTGTCCCCAGCCCAGTGGGCATTGGAGGTGCCAGGGGAGTCAGGGTTGTAGC
CAAGACGCCCCCGCACGGGGAGGGTTGGGAAGGGGGTGCAGGAAGCTCAACCCCTCTGGGCACCAACCCTGCATTGCAGGTTGGCACCTTAC
TTCCCTGGGATCCCCAGAGTTGGTCCAAGGAGGGAGAGTGGGTTCTCAATACGGTACCAAAGATATAATCACCTAGGTTTACAAATATTTTTAG
GACTCACGTTAACTCACATTTATACAGCAGAAATGCTATTTTGTATGCTGTTGAGTTTTTCTATCTGTGTACTTTTTTTTAAGGGAAAGATTTTAAT
ATTAAACCTGGTGCTTCTCACTCACA
SEQ ID NO: 3
Protein name: Wild-type DDR2 protein (DDR2_NP_001014796:)
Origin: Human
MILIPRMLLVLFLLLPILSSAKAQVNPAICRYPLGMSGGQIPDEDITASSQWSESTAAKYGRLDSEEGDGAWCPEIPVEPDDLKEFLQIDLHTLHFI
TLVGTQGRHAGGHGIEFAPMYKINYSRDGTRWISWRNRHGKQVLDGNSNPYDIFLKDLEPPIVARFVRFIPVTDHSMNVCMRVELYGCVWLDGLVSY
NAPAGQQFVLPGGSIIYLNDSVYDGAVGYSMTEGLGQLTDGVSGLDDFTQTHEYHVWPGYDYVGWRNESATNGYIEIMFEFDRIRNFTTMKVHCNNM
FAKGVKIFKEVQCYFRSEASEWEPNAISFPLVLDDVNPSARFVTVPLHHRMASAIKCQYHFADTWMMFSEITFQSDAAMYNNSEALPTSPMAPTTYD
PMLKVDDSNTRILIGCLVAIIFILLAIIVIILWRQFWQKMLEKASRRMLDDEMTVSLSLPSDSSMFNNNRSSSPSEQGSNSTYDRIFPLRPDYQEPS
RLIRKLPEFAPGEEESGCSGVVKPVQPSGPEGVPHYAEADIVNLQGVTGGNTYSVPAVTMDLLSGKDVAVEEFPRKLLTFKEKLGEGQFGEVHLCEV
EGMEKFKDKDFALDVSANQPVLVAVKMLRADANKNARNDFLKEIKIMSRLKDPNIIHLLAVCITDDPLCMITEYMENGDLNQFLSRHEPPNSSSSDV
RTVSYTNLKFMATQIASGMKYLSSLNFVHRDLATRNCLVGKNYTIKIADFGMSRNLYSGDYYRIQGRAVLPIRWMSWESILLGKFTTASDVWAFGVT
LWETFTFCQEQPYSQLSDEQVIENTGEFFRDQGRQTYLPQPAICPDSVYKLMLSCWRRDTKNRPSFQEIHLLLLQQGDE
SEQ ID NO: 4
Protein name: Wild-type DDR2 protein (DDR2_NM_001014796:)
Origin: Human
CAAAGGCATCTTGCATCAGCCTGTGGATGTATGCCTACCACCGGGCTCCTTCACCAGCAAAGTGGAAAAAGAAGCGTTTCACAACAAATTCTTC
TTTTTGGGTTGGGGAAACGCAGTGGATTATAGCTCTGTTTTCTTCTTTCCAAAACTGTGCACCCCTGGATGAAACCTCCATCAAGGGAGACCTG
CAAGTTGCCTGGGGTTCAGTGCTCTAGAAAGTTCCAAGGTTTGTGGCTTGAATTATTCTAAAGAAGCTGAAATAATTGAAGAGAAGCAGAGGC
CAGCTGTTTTTGAGGATCCTGCTCCACAGAGAATGCTCTGCACCCGTTGATATGCCTCCCAGGACCCAGAGGGAGACTGTAGCCTCATTTCTGT
GGAGACCTTTGGCTGGACTCTCCTGGCTCTCCCAGAGACTCCAGTTCCAACACCATCTTCTGAGATGATCCTGATTCCCAGAATGCTCTTGGTGC
TGTTCCTGCTGCTGCCTATCTTGAGTTCTGCAAAAGCTCAGGTTAATCCAGCTATATGCCGCTATCCTCTGGGCATGTCAGGAGGCCAGATTCCA
GATGAGGACATCACAGCTTCCAGTCAGTGGTCAGAGTCCACAGCTGCCAAATATGGAAGGCTGGACTCAGAAGAAGGGGATGGAGCCTGGT
GCCCTGAGATTCCAGTGGAACCTGATGACCTGAAGGAGTTTCTGCAGATTGACTTGCACACCCTCCATTTTATCACTCTGGTGGGGACCCAGGG
GCGCCATGCAGGAGGTCATGGCATCGAGTTTGCCCCCATGTACAAGATCAATTACAGTCGGGATGGCACTCGCTGGATCTCTTGGCGGAACCG
TCATGGGAAACAGGTGCTGGATGGAAATAGTAACCCCTATGACATTTTCCTAAAGGACTTGGAGCCGCCCATTGTAGCCAGATTTGTCCGGTTC
ATTCCAGTCACCGACCACTCCATGAATGTGTGTATGAGAGTGGAGCTTTACGGCTGTGTCTGGCTAGATGGCTTGGTGTCTTACAATGCTCCAG
CTGGGCAGCAGTTTGTACTCCCTGGAGGTTCCATCATTTATCTGAATGATTCTGTCTATGATGGAGCTGTTGGATACAGCATGACAGAAGGGCT
AGGCCAATTGACCGATGGTGTGTCTGGCCTGGACGATTTCACCCAGACCCATGAATACCACGTGTGGCCCGGCTATGACTATGTGGGCTGGCG
GAACGAGAGTGCCACCAATGGCTACATTGAGATCATGTTTGAATTTGACCGCATCAGGAATTTCACTACCATGAAGGTCCACTGCAACAACATG
TTTGCTAAAGGTGTGAAGATCTTTAAGGAGGTACAGTGCTACTTCCGCTCTGAAGCCAGTGAGTGGGAACCTAATGCCATTTCCTTCCCCCTTG
TCCTGGATGACGTCAACCCCAGTGCTCGGTTTGTCACGGTGCCTCTCCACCACCGAATGGCCAGTGCCATCAAGTGTCAATACCATTITGCAGA
TACCTGGATGATGTTCAGTGAGATCACCTTCCAATCAGATGCTGCAATGTACAACAACTCTGAAGCCCTGCCCACCTCTCCTATGGCACCCACAA
CCTATGATCCAATGCTTAAAGTTGATGACAGCAACACTCGGATCCTGATTGGCTGCTTGGTGGCCATCATCTTTATCCTCCTGGCCATCATTGTC
ATCATCCTCTGGAGGCAGTTCTGGCAGAAAATGCTGGAGAAGGCTTCTCGGAGGATGCTGGATGATGAAATGACAGTCAGCCTTTCCCTGCCA
AGTGATTCTAGCATGTTCAACAATAACCGCTCCTCATCACCTAGTGAACAAGGGTCCAACTCGACTTACGATCGCATCTTTCCCCTTCGCCCTGA
CTACCAGGAGCCATCCAGGCTGATACGAAAACTCCCAGAATTTGCTCCAGGGGAGGAGGAGTCAGGCTGCAGCGGTGTTGTGAAGCCAGTCC
AGCCCAGTGGCCCTGAGGGGGTGCCCCACTATGCAGAGGCTGACATAGTGAACCTCCAAGGAGTGACAGGAGGCAACACATACTCAGTGCCT
GCCGTCACCATGGACCTGCTCTCAGGAAAAGATGTGGCTGTGGAGGAGTTCCCCAGGAAACTCCTAACTTTCAAAGAGAAGCTGGGAGAAGG
ACAGTTTGGGGAGGTTCATCTCTGTGAAGTGGAGGGAATGGAAAAATTCAAAGACAAAGATTTTGCCCTAGATGTCAGTGCCAACCAGCCTGT
CCTGGTGGCTGTGAAAATGCTCCGAGCAGATGCCAACAAGAATGCCAGGAATGATTTTCTTAAGGAGATAAAGATCATGTCTCGGCTCAAGGA
CCCAAACATCATCCATCTATTAGCTGTGTGTATCACTGATGACCCTCTCTGTATGATCACTGAATACATGGAGAATGGAGATCTCAATCAGTTTC
TTTCCCGCCACGAGCCCCCTAATTCTTCCTCCAGCGATGTACGCACTGTCAGTTACACCAATCTGAAGTTTATGGCTACCCAAATTGCCTCTGGC
ATGAAGTACCTTTCCTCTCTTAATTTTGTTCACCGAGATCTGGCCACACGAAACTGTTTAGTGGGTAAGAACTACACAATCAAGATAGCTGACTT
TGGAATGAGCAGGAACCTGTACAGTGGTGACTATTACCGGATCCAGGGCCGGGCAGTGCTCCCTATCCGCTGGATGTCTTGGGAGAGTATCTT
GCTGGGCAAGTTCACTACAGCAAGTGATGTGTGGGCCTTTGGGGTTACTTTGTGGGAGACTTTCACCTTTTGTCAAGAACAGCCCTATTCCCAG
CTGTCAGATGAACAGGTTATTGAGAATACTGGAGAGTTCTTCCGAGACCAAGGGAGGCAGACTTACCTCCCTCAACCAGCCATTTGTCCTGACT
CTGTGTATAAGCTGATGCTCAGCTGCTGGAGAAGAGATACGAAGAACCGTCCCTCATTCCAAGAAATCCACCTTCTGCTCCTTCAACAAGGCGA
CGAGTGATGCTGTCAGTGCCTGGCCATGTTCCTACGGCTCAGGTCCTCCCTACAAGACCTACCACTCACCCATGCCTATGCCACTCCATCTGGAC
ATTTAATGAAACTGAGAGACAGAGGCTTGTTTGCTTTGCCCTCTTTTCCTGGTCACCCCCACTCCCTACCCCTGACTCATATACACTTTTTTTTTTT
TTTACATTAAAGAACTAAAAAAGGAAAAAAAAAAGCCTAGGGCAGATACAATCTAGTAAAAGAAAATCTTTGATATACCAAAGTGTTGGATAAC
AAAGGCTAGAAAATTCAGATAATTTATAAAGGTTAACTATACTTGTGCTTATAAATGTGCAGATTCTACAATATTTTTCCATGTCATTCTAAAAGA
ATCTTCAGAAAGAAAAACTTGAAGAATACTAATGTCTTGGGAAACATGAGATTAAGTTTAGGGAAAACACTTGATAGATGTGGAGAATCTGAG
GACTCAGAATTCAGCAACTAATCACAGGTGGTTTTCTAATTTGGCCTCTGGACATGTCTCACTGTTTGTATTTCTCTCTCCTGTCAAAGTGAATGA
TATATTCTTGAAAACCCCCAATTTCTTGAAATGGGTGTTCTGTTCATTCATGGGCAGGGGTCAGGATTGAAGTTCATATATGAAACAACTGGGG
ATGTAGATAGGAAAGAATGTCTGCTGCAAGAGTGTATGAAGGGGGATTGTCATTTACAAAAAAAAAGTACCTCATGGATGGAAGGATTCATGA
ATAGATAATGGACATAGGAAAGGAGGTCAATGGAGGACACATAACAGACATGCTATCCACCATTTATTTGTGATTTTGTAAGAGGGTTCTCTTC
TTTGTCTTGTTCAGATATTTTCATGTGTGTGTTTTAGTGAGAATCTGAGTTTTTAATCAGTAAAGTCTGATGTTTTGATATCTTGATGTTTTGATG
GTAGACTCTTGAGTTCATCTTGTCCAAATCTTCTAGCATTTTCAAGGATGAAGCCTCTGTGGGATCTTTGGGCTTGGGGCTGAATTTCTCAGGCA
TAAATACCTAATGGCCTTGTCTAAGAGGGAATAGATCTGTTCTGAGTGTCCCCAAAAAAGTTGTCCTAGGAACTGATGTGGAGAAGTTACAGG
GAGACTGATTTCCATTCCTTATTGGGGAAAACGAAGCACTCAGAACATACAAGGATAAATGGGCTGCCACCAGAGGTGAGGAGTCTCTTGTCA
CTGGAGAGGTTAAGATAGAGGCTCAAGAACAATGCAAGGAAAATGGAGGTTTTCAAACACAAAGTGGATGGTGGTATGAGACAATGTTTAAT
GTCCTTCTAACTCCGAGAGTCCTTGATTCTGGAGAGGCACAGGATGAACATCTGTTGCTACTGCTGAGTATACACACTCTTGCCTCTGAGATGG
CGTGACCAGCAGAAAAGAAAGCTCAGGGCTGTGGCTTCAGAATTCATGGAAACAGAGCCACTGTCAAGAAGGAATCTGCTCAGAGCAGATTC
TGAGTTTTCACACTCCACAAATCCTCAGATTTAGTGGGAAGCTGGAAAAAGGAACTCTTAATTCCAATTGAGGAGAAACAAGAAAACATTACAA
ATCAGTTTCCCAAGCTGAGAAGGATTAGCCTTGACCCCTTGGGTCTGCCTCTGTTGCCCTCACCAGTTTACCTTCTACAACAGTTTCTCAGGAAC
ATGTGTATTTCCCCATTCAGCTTTCAGCATTGCTCCACGCCACAGCATAAGCATAGATCCCAAGTCCACAGGCTCCATTTTGCAGGTCATCTTCT
GATCCTAGCAAATGTCCTTTCCCCATAGTTGTCCTATGCCTTTGGGCTTTAGTCTATCCCAGGACTAACTGTGGAGAAATCATTGGTTTGAGAGT
CAAGAGAGCATTGGTTTGGGAGCTTTAATCCTCTTTCTGCTTCACACTAAGTGTGTCATCTTGGCTAAATCACTTGGTCTTTCTGCATTTTGTTTT
CTTATTTATAGGATGAGGAAATTAGATTAAATGGTTTTGAGGTCCTTTCTTGTTCTGATATGTCCAGTACTCACTGGAAAATTGGATCTATAACT
GATGGGTTTAGTAATCTGGTCATTTCTTGCTCTGAAAATTGTAGTCAGCAAAAGAGATCATGGAAGAAATCACTGTAATGGTAGTAATAGTAAC
ACATGCCATTTGTATTGTGCCTTAGGTTTACCAGGTGTTTCCAAATACATTAGCATATTTGATATGTGCAGGACTAGATACCTTGGGACCTGCCA
CACTCCACTTTCAAGATATGTATTAGCTTCATTAGAATTAAAGGGACTTGAACTCAGGACCTGCAGCCTATTCTTCTTTATCCACATGTCTCTGGT
AGGGCTACATCCAGATCACACCATGACTTCTTATAGAGCAAGAGAAAATAATATTATTATATCTTCCTTTGCCTAAAATCTCTCCACTTATTCTTTT
TTATGATTCTGCACCAGTTCACTGGGTTATTCTATGATTCCATATTTTTTTTAAAAAAAATCATATTTAAAATGAACTTACAATGTCTGAATTTTCC
TGGCCTTGAGTCACAGAAGTAATATGTTTCAGATGGCTGCCCAATATGTATTATCATGTAATACATATCTGTGTCCTTTTCTGGGATGAGGAAG
GCTTCAACTTCTGGCACTGAGAACTTTGTATTACAGACACAGGTTTAGTTTCTAGCTTAGTCATGCCTTTAGAGTTTAGTAGCACAAATCCATGC
AACCCAACAGAATACATGGTGAGGGCCTAGTATGTAGAATTTGAAAGTAGTTCAAATTTGAATTAGAAGAATGAAGCTAGGACTTATTTGGAA
AAGGAGATAGAAAAAAAATGATCAGAAACTGTGGGGCCTTATTACCTTTGCAGTAAGTTATCTTCTTCATGATATATGTGAATTATTTTATGTGC
AGATTGTGTTTTGGGATTGTCAGATCTAAACTTATATTCTTGCTGGCTAATGTGCTGATAGCCAGGTCTGAAACTTGATGTGCTATCCAGACACA
TATGATCAGAAAAGATCTAGAGTGCAAAGAGGTGCCTGAGGAGCAAAATGTGGTITTAATGTTGTGGAAAGATCACTTGCAAGTATATAAGAC
TGTATAAAGAAGAGGACTGTGTGCAAGTGGGGTGAAAAAAAAGGATACGTGAATGTGCATATGACTGAATAGGGAGGAAGGTCAGGGCTAG
AAAGGAGGCTACATAAAAAGGGGCAATGGAGAATGCACAGGAAAGACACAGGGGAAGGTCAAGTCGAGCAAGGTAGAAACAGGAGTAGCT
AGAGCCATTGGGAATCCATTTTGAAACAAGAAGGAGTTTTGAAAGGGAATAGGAAAGTAAGTGTCTTGAAGTAAAAGATAAATATGGATGGA
GAAAGAAGAAATTCTGGATGATAGAGATGATAAAAATATTTATTAAGAAATGAAGTCAGGTTCAGTGTATGAAATGGAAAGGAATTTTTCAGA
ATTTTAAGAAAGGGGAAGTTCCTCTTGGAAAAGATATAGCAACCATCGGGGAATGACCTTTTCATTTCAGAAGTGGATGAGGAAGGTGGTGTG
AGCATCAGGTATATTCTGGACCATTTCAAGTGCTGGTGAGAAGAAAGGAACTCTTTGCCTGAACTGGGCTTGGTTTTCCAAGTGCTGCTTTGGA
AATGAAGACCCAGAGATGCAGAGCTTATGGTAGTTCATAAATCTTCATGTTCTATTATCTTTCATCTGCCAATAAAGTTCATTTTCAATAATGTCC
ACCATTGCTGTGCCCAGAATAACCACAGGCAAACATCAAAACAATACGCATAAGTTAGACAAGATTAAATCTTGTCTGATATCTGCACAAACAG
ATATGCACCATGTTGGAAACATGTGTTTTCCTAGTCCCATCCAGGCTTCCCACAAGAAAGCCATGATGTGGGTCTAAACCATATGTTTTGAGTAA
AGGAGAATAGAAGAAGGGGAGTGTCCGCAAAATGGAAAGAGATGAAGATGTTCCAAGGAAGTATGCTGAAACAGAACAGTGAATGTTTTGC
CCAAAACTACAAAAATAAAAGAAAAAAAGAAAATTGCAATACATGGCTACTAAGTCTTTGATCATAAGTCGAATTTATAGACCTGGAATTTGCC
ATCCTAGTCTTTCCTTTTTAGTAAGACTTCTGTCCTCTGGCAGTGCATATGGTAGGTCTCTAATGTTTCTTCATCTCCAGGAAGATGCAGATCCTT
ATTTTTGCTGGGAAATCCTTCTAAATAGAAATGTAACATTTTTATAAAAACAGATTAATGTGTTTTTCACTTAGTAAATGTTTTCAAGAGCTGAAT
TGAGAAGGAAAGAGACTGGAGTGGTTAATGGTGATTTGATTTCTGGCATTCTGAGTTTTCTGCTACAATTAGCTGCATTACTTGGTGCCAAAGA
GCAGTGGGGAATTGTTGAGTTGCTGTATCCTTTAAAAAAAAACAAAAAACTTGTTATTTTGAAAGAACTTAAGGCTCACAAGATGTTACAAAAA
TAGTAGAGTGGCCTTACCCTAGATCCAGTTTTCCCCATTTATAACATTTCACTTAGTCCATTTTCGGAACCAGAAAATTAACATTGGCATAATGCT
ATTAACTAAACTACAGACCTTTTTTCAATTTCGCCAGTTTTTCCACACATATTCATTTAGTTGCTGGATACTTTTAATTCTTGCTGATTTGTAAACT
GGCCTTGCTTGGATACAACAGGAAAGATACTATCTGGATAAAGTTCTACAGTTTTAGAGAGACTATTAACACATTAATGTGTTCCTTTGTCATGA
GCAATACCCTGCCTACACTGCTTCTAAATTTTCTGATTTGTTTGGCTGTTTGGCATCTGAAACAATCCAAGACAAACTTAGAAAGATTAGGCAAC
ACAAAACACAGTAAGACCTGTTCATAGCTTGTTGTCTAGAAAACCAGGTAGCAGGATATTCTAGATGCTTCCTGCTGCTTCTACGTGAGTAGGA
TTAGCACTGGGGACAAAATAAGGAGTTTAGAGTAAACCAGTATTTCAGTCAAGAGTTAGTTGGCACTTAGTTAATGGCACTGGAAATAGCTTGT
GGAGAGAATAGAATACAATGGTATAGACTCCTAATGTTTGATAAAATACTATTTTCAGAGTGGTAGAGAGGTTTTATTTGCCTAAATAGCCGTT
ATTAAATGGAATAACAACCACATTAGACCAAATTAATTGCAAACACAGCGGCAACCTGGGGAGAAGTTGAAACTCCAGTTTTGTGGATTACAGT
TTTGAGTITTATGATTGACATTTTTAAGTCCCCTATTTAAGGGGTCAAGATTATAACAATGTGTGTCTTACTAAGTTTCTAGGTCATTGTGAGCAC
TTGATAAATATTTGCTGAATGTTGTTTTTTTGAATGAACATAAGATAGAAACAAAAACTTCTCATCCAGTTAACTAGAGTGAATGTAGGGAGAAT
TGTTTTGCTTGTAACATGGAGAGTTTATTTTCAAGTGAGGAAAGAGAAAAAAATTACTCAGACTTGTTCCTGGTGAAGTGCATTCTCTGTTTGTA
TACTTTTTGATGGAGAAATTGATCTATAGAACTGCTTAATTTTTTGAGGCATTTAGACAGCAATGAAAGGTAGTTCTCCACAGGACACCGAATCA
AAAGGAGAGACCAGACTCTGGCCTCATACCCAGCCTATTTGAAACAAGCTATCTAGTTTCTCCTGCAGACACCTTGTCAACAACATGCAACAGT
GTCAGGTGCCTTGCAGGAAAATAATCTGAGTCCCAAGCTAGCCTGTGCTCATCCACAATCACAATGAACATGTCAAGGAAGAATTTGCAGAGA
CTCAAGGGAAGCACAATGGGATAAGGTAATCACTTTCAGTGAAAAACTGTTTTCTTGAAAACAGGCTTGGACACAATTGAAAGCTGGCTTCCTG
CAAACACACCAAGAGTCTGTAATCTAGCCTATCCATTATATGTCCTTTATTATTCATGATATCCTATTCTTCTACCTTGTTGCCTGGTAACTTTTTC
TGAGGACTGAGTTTCTGCAGCGATGTGGTGCACTCTTCCTGTGATGAGGAAACATCTGGGCCCCCTTCTGCAGGCTTTGGAAGATGATGTGTCT
TGTCAAGGGGTAAAGGGCAAATGGATTTAATTTCTGCTTAAAACTATCATAGACGTTCCAAATAGAATATGTAAAATTTCTCTGTATTAGAAAAA
GAAACGTGATACCAATTGTATATTTTCTTTTCTTTATTTATTCTCTGTAAGTCTGTCAGATGATAAATTGTAAATAACAATGATTAAAGAGTCATG
CTACTGATGGATCTCCCTTTCTGTATAAACAGTGCCAGTTCTGGGCTTTGTAACCTTTGCTCTTTATAGTCTTTCATTCCTGGGGAAGTGATGGG
GCATGGGCCCAGAGCTGGGGTGTATGTGGTATGGACACCTGTTTGTGGGGCTTTCCAGCAAAGGATTATTTAAATAGACCTCTAACATATGAG
TTGACTGTTTATTGGGAAGAAAGCATCTTGGTCTCTGACATATCCAAACATACGAGACACTGGGATTTTACGTCCTCACATTAATTAGTCCAGTT
CTGGGGAATCCAGTCTAGAATTAACTGGTGATCCCTTATCGTTATGGTTACAGACTTGTCTTGTTTGATACACAGAAATCCTTTTTAAATCCAAAT
ATAGTCTGTCTCAGACTACCTGCACATGCACAAACCAAAAAAAACTATGAAAACCAGAATTTAGACTCAGTCATTATACTAGAGATTAGAATGA
AACGACCCCAAACAACCCATTTCTTACCTGGTCTCTGAGAATAAGTTTATACTTTTAGTTTTCTGAGAATATTTCTCAGAATATGTGAGTTTTCTG
AGCACATTTTTCAGCATGGGGTATGGTAGATAAATCACAGGGCCAAGGTTTGGCAGGGATAGATGGGATCATTGTGTACCCTATTGTTCTTCTG
ATTTCCAGGAGAACAGAATGAGCCCATGCAAAACATAAACTTATGATGATTAAAAAAAACACACCTATCCATTCACTCATCAATAAAAACATATT
ATGATGGTTATCAAGCTGTGTCCTATGAGTGATAAAATATTTGTAAAATATAAAATTAAATGGCATCTATTTTGAACTCTA

1. (canceled)

2. A method of treating or preventing vascularisation in one or both corneas of a subject, the method comprising topically administering an effective amount of a topical composition comprising dasatinib to one or both eyes of the subject.

3. (canceled)

4. The method as claimed in claim 2, wherein
the vascularisation is associated with or caused by the aberrant expression of a receptor tyrosine kinase (RTK) in one or both eyes of the subject.

5. The method as claimed in claim 4, wherein the RTK is:
(i) PDGFRβ (Platelet Derived Growth Factor Receptor-beta), or
(ii) DDR2 (Discoidin Domain-containing Receptor 2).

6. The method as claimed in claim 5, wherein the vascularisation is due to:
(i) one or more mutations in the PDGFRβ gene; or
(ii) one or more mutations in the DDR2 gene.

7. The method as claimed in claim 6, wherein the mutation is a temperature-sensitive or a non-temperature-sensitive mutation.

8. The method as claimed in claim 7, wherein the temperature-sensitive mutation is:
(i) N666Y in the PDGFRβ gene; or
(ii) Y740C in the DDR2 gene.

9. The method as claimed in claim 7, wherein the non-temperature-sensitive mutation is:
(i) S548S, N666S, V665A, N666H, W566R and P584R in the PDGFRβ gene;
(ii) L610P in the DDR2 gene.

10. The method as claimed in claim 2, wherein the subject has:
(i) ocular pterygium-digital keloid dysplasia (OPDKD),
(ii) Warburg-Cinotti syndrome (WCS), or
(iii) Penttinen syndrome.

11. The method as claimed in claim 2, wherein the topical composition is administered to one or both of the subject's eyes for 5-7 hours.

12. A method of diagnosing OPDKD in a subject, the method comprising the steps:
(a) detecting, from a biological sample obtained from the subject, whether the subject's PDGFRβ gene has the mutation N666Y and/or SS48Y; and
(b) diagnosing the subject with OPDKD if the presence of the N666Y and/or S548Y mutation in the biological sample is detected; and optionally,
(c) administering an effective amount of an RTK inhibitor to the diagnosed subject.

13. A pharmaceutical composition comprising dasatinib, wherein the pharmaceutical composition is in the form of a topical composition for administration to the eyes, optionally together with one or more ophthalmically-acceptable excipients, diluents or carriers.

14. A pharmaceutical composition as claimed in claim 13, wherein the dasatinib has a concentration of 0.01-100 μM.

15. A pharmaceutical composition as claimed in claim 13, wherein the dasatinib has a concentration of 20-100 μM.

16. The pharmaceutical composition as claimed claim 13, wherein the composition is in the form of a solution, a suspension, an ointment, a gel or a foam.

17. The pharmaceutical composition as claimed in claim 13, wherein the dasatinib is in the form of a nanoparticle-based ocular delivery system.

18. (canceled)

19. The method as claimed in claim 5,
wherein the vascularisation is due to:
(i) one or more mutations in the PDGFRβ gene which leads to increased activation of the PDGFRβ protein;
(ii) one or more mutations in the DDR2 gene which leads to increased activation of the DDR2 protein.

20. The method as claimed in claim 12, wherein the RTK inhibitor is dasatinib.

21. The pharmaceutical composition as claimed in claim 17, wherein the dasatinib is in the form of a nanoparticle-based ocular delivery system selected from the group consisting of nanospheres, nanocapsules, liposomes, hydrogels, dendrimers, nanoparticles and nanomicelles.
